(19) United States
(12) Patent Application Publication
(10) Pub. No.: US 2025/0333440 A1
(43) Pub. Date: Oct. 30, 2025

(54) Azole Tripeptides as Vasodilators

(71) Applicant: University of Kansas, Lawrence, KS (US)

(72) Inventors: Steven Bloom, Lawrence, KS (US); Allen Alonso Rodriguez Ugalde, Lawrence, KS (US)

(21) Appl. No.: 19/190,047

(22) Filed: Apr. 25, 2025

Related U.S. Application Data
(60) Provisional application No. 63/639,361, filed on Apr. 26, 2024.

(51) Int. Cl. C07K 5/097; A61K 38/00; A61P 9/08; C07K 1/06

(52) U.S. Cl. CPC C07K 5/0821; A61P 9/08; C07K 1/068; A61K 38/00

(57) ABSTRACT

Azole tripeptides may function as vasodilators. These compounds may contain three azole groups connected in a peptide-like structure. The azole groups may be monocyclic or fused bicyclic heteroaryl groups. Each azole group may be substituted or unsubstituted. The compounds may include various end cap moieties. A pharmaceutical composition may include an azole tripeptide compound and a pharmaceutically acceptable carrier. Methods of vasodilation may include administering an azole tripeptide compound to a subject in a therapeutically effective amount. The administration may cause vasodilation in the subject. The vasodilation may provide treatment for various conditions. The azole tripeptide compounds may be synthesized through multi-step chemical processes.

### CROSS-REFERENCE TO RELATED APPLICATIONS

This patent application claims priority to U.S. Provisional Application No. 63/639,361 filed Apr. 26, 2024, which provisional application is incorporated herein by specific reference in its entirety.

### U.S. GOVERNMENT RIGHTS

This invention was made with government support under GM 147169 awarded by the National Institutes of Health. The government has certain rights in the invention.

### SEQUENCE LISTING STATEMENT

The Sequence Listing submitted as the XML file named K 1262.10107US02.xml, created on 2025-04-25, and having a size of 1,941 bytes, is hereby incorporated by reference in its entirety.

### BACKGROUND

### Field

The present disclosure relates to compounds and compositions for use as vasodilators. More particularly, the compounds can include azole tripeptides.

### Description of Related Art

Vasodilators represent a well-established class of therapeutic agents that function primarily by relaxing the smooth muscle within blood vessel walls, thereby reducing vascular resistance and promoting increased blood flow. These agents are commonly employed in the treatment of conditions such as hypertension, angina pectoris, heart failure, and peripheral vascular diseases. The state of the art includes a variety of vasodilator types, including direct-acting agents (e.g., hydralazine), nitric oxide donors (e.g., nitroglycerin, isosorbide dinitrate), calcium channel blockers (e.g., amlodipine, nifedipine), angiotensin-converting enzyme (ACE) inhibitors, and phosphodiesterase inhibitors (e.g., sildenafil). Additionally, certain prostaglandins and endothelin receptor antagonists have been developed for more specialized indications. Advances in the field continue to focus on improving the selectivity, duration of action, and side effect profiles of these agents, as well as exploring targeted delivery systems and combination therapies to enhance clinical efficacy.

Despite the widespread use of vasodilators in clinical practice, current therapies are often limited by several significant drawbacks. Many vasodilators lack target specificity, leading to systemic hypotension and associated side effects such as dizziness, headaches, and reflex tachycardia. Tolerance development, particularly with nitrate-based agents, reduces long-term efficacy and necessitates dosing strategies that limit continuous use. Additionally, some vasodilators exhibit poor pharmacokinetic profiles, including short half-lives and low bioavailability, requiring frequent dosing or complex delivery systems. These limitations underscore the need for improved vasodilator compounds or formulations that offer enhanced selectivity, sustained activity, and reduced adverse effects.

Thus, there is a need for improved compounds and therapeutic compositions that can be used as vasodilators.

### SUMMARY

In some embodiments, a vasodilating compound can be a azole tripeptide that can include a structure of Formula 1, Formula 1A, or Formula 1B, or derivative thereof, prodrug thereof, salt thereof, stereoisomer thereof, or having any chirality at any chiral center, or tautomer, polymorph, solvate, or combination thereof,

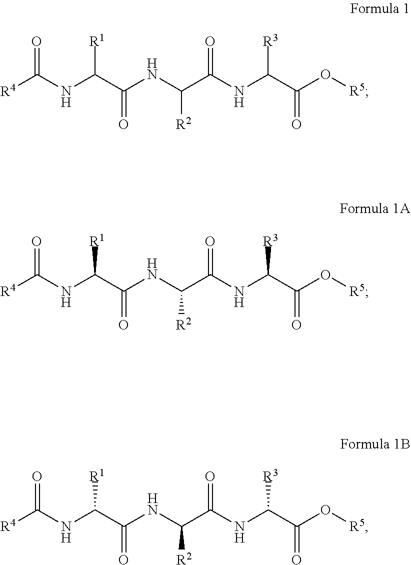

In Formula 1, Formula 1A, or Formula 1B, R¹, R², and R³ each independently include a substituent having at least one azole group that is substituted or unsubstituted, and R⁴ and R⁵ each independently include an end cap moiety.

In some embodiments, each azole group of the tripeptide is independently selected from pyrrole, imidazole, pyrazole, triazole (1,2,3- and 1,2,4-isomers), tetrazole, oxazole, isoxazole, thiazole, isothiazole, and benzofused analogs such as benzoxazole, benzimidazole, benzothiazole, indole, indazole, carbazole, isoindole, benzotriazole, and combinations thereof.

In some embodiments, a composition can include the vasodilating compound and a carrier. A pharmaceutical composition can include a pharmaceutically acceptable carrier.

In some embodiments, a method of vasodilation can include providing a subject; and administering an embodiment of a vasodilating azole tripeptide compound to the subject in a therapeutically effective amount to cause vasodilation in the subject.

In some embodiments, a method of treatment of a condition in a subject can include administering an embodiment of the vasodilating azole tripeptide compound to the subject in a therapeutically effective amount to cause vasodilation in the subject to provide the treatment of the condition. The vasodilating compound can be an azole tripeptide, and the condition can be any condition treated with vasodilation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A illustrates reaction Scheme 1.

FIG. 1B illustrates reaction Scheme 2.

FIG. 1C illustrates reaction Scheme 3.

FIG. 1D illustrates reaction Scheme 4.

FIG. 1E illustrates reaction Scheme 5, which results in Compound 1.

FIG. 2A includes an image that shows a large number of branching points.

FIG. 2B includes an image that shows vascularization, with vascular density and length density.

FIG. 2C includes an image that shows the vessel thickness, such as average vessel diameter.

FIG. 3A illustrates the vascular density that results from exposure to the Compound 1 tripeptide against controls and angiogenesis modulators, razuprotafib and NLLMAAS heptapeptide.

FIG. 3B illustrates the number of branches that results from exposure to the Compound 1 tripeptide against controls and angiogenesis modulators, razuprotafib and NLLMAAS heptapeptide.

FIG. 3C illustrates the vessel diameter that results from exposure to the Compound 1 tripeptide against controls and angiogenesis modulators.

FIG. 3D illustrates the vessel length density that results from exposure to the Compound 1 tripeptide against controls and angiogenesis modulators, razuprotafib and NLLMAAS heptapeptide.

FIG. 4A illustrates the vessel diameter that results from exposure to the a 4 μM and 40 μM of Compound 1 tripeptide.

FIG. 4B illustrates the vessel length density that results from exposure to the a 4 μM and 40 μM of Compound 1 tripeptide.

FIG. 5A illustrates the vessel diameter that results from exposure to the Compound 1 tripeptide against controls and angiogenesis modulators, VEGF, ANG-1, and ANtiVE-PTP mAB.

FIG. 5B illustrates the vessel length density that results from exposure to the Compound 1 tripeptide against controls and angiogenesis modulators, VEGF, ANG-1, and ANtiVE-PTP mAB.

FIG. 6A illustrates the vessel diameter that results from exposure to the Compound 1 tripeptide against controls and vasodilators, amlodipine, isosorbide, and prazosin.

FIG. 6B illustrates the vessel length density that results from exposure to the Compound 1 tripeptide against controls and vasodilators, amlodipine, isosorbide, and prazosin.

FIG. 6C illustrates vascular density that results from exposure to the Compound 1 tripeptide against controls and vasodilators, amlodipine, isosorbide, and prazosin.

FIG. 6D illustrates the number of branches for Compound 1 versus the DMSO control.

FIG. 6E illustrates junction voxels for Compound 1 versus the DMSO control.

FIG. 7A shows a graph of the vessel diameter that results for control (flat) versus the tripeptide at 40 μM (having peak) over time.

FIG. 7B shows a graph of the vessel length density that results for control (flat) versus the tripeptide at 40 μM (having peak) over time.

FIG. 8A illustrates Scheme 1A, which is related to Scheme 1.

FIG. 8B illustrates Scheme 2A, which is related to Scheme 2.

FIG. 8C illustrates Scheme 3A, which is related to Scheme 3.

FIG. 8D illustrates Scheme 4A, which is related to Scheme 4.

FIG. 8E illustrates Scheme 5A, which is related to Scheme 5, which results in the azole tripeptide derivative.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present invention provides azole tripeptides that have bioactivity as vasodilators and which can be synthesized with stereospecific chemistry. The vasodilators can include one or more azole tripeptides. An example azole tripeptide is methyl (S)-2-((S)-2-((S)-3-(1H-benzo[d][1,2,3]triazol-1-yl)-2-(2,2,2-trifluoroacetamido)propanamido)-3-(5-methyl-1H-tetrazol-1-yl)propanamido)-3-(1H-1,2,4-triazol-1-yl)propanoate (Compound 1) as shown below.

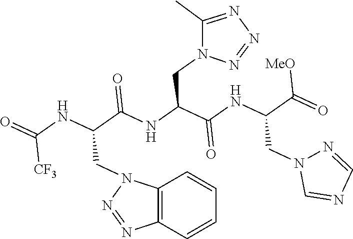 [SMILES: COC(=O)[C@H](Cn1cncn1)NC(=O)[C@H](Cn1nnnc1C)NC(=O)[C@H](Cn1nnc2ccccc21)NC(=O)C(F)(F)F]

methyl (S)-2-((S)-2-((S)-3-(1H-benzo[d][1,2,3]triazol-1-yl)-2-(2,2,2-trifluoroacetamido)propanamido)-3-(5-methyl-1H-tetrazol-1-yl)propanamido)-3-(1H-1,2,4-triazol-1-yl)propanoate, (Compound 1)

This azole tripeptide has demonstrated vasorelaxant activity in an in-vivo chick chorioallantoic membrane assay (CAM), as shown herein, which indicates functionality as a vasodilator. Additionally, derivatives of this azole tripeptide can also be used as vasodilators, where the chemical structure can be derivatized to maintain vasodilation functionality. However, it should be recognized that different azoles can be placed at each azole peptide.

For vasodilators, the functionality can include an increase in vessel diameter and a decrease in the vessel-length density. The latter is considered to be consistent with widening of the vessel, which then causes vessel shortening. As shown herein, it has been found that Compound 1 indeed increases vessel diameter in Day 6 Brown Leghorn embryos by 124% (p<0.001) and decreases vessel length density by −17% (p<0.05) compared with DMSO control (e.g., as calculated with the NIH ImageJ Software at 24 hr. post single-dose (40 μM; 0.91 mg/kg)).

The stereochemistry of tripeptides composed of L-amino acids (designated as the “S” configuration at the α-carbon for most standard amino acids) plays a critical role in determining their three-dimensional structure, biological activity, and interaction with enzymes and receptors. When all three residues in a tripeptide adopt the L-configuration, the resulting backbone conformation favors specific dihedral angle arrangements (φ, ψ) consistent with right-handed α-helical or β-sheet structures commonly found in natural proteins. This uniform stereochemistry contributes to predictable folding patterns and influences hydrogen bonding potential, side chain orientation, and overall molecular stability. Additionally, the exclusive use of L-amino acids allows these tripeptides to be efficiently recognized and processed by endogenous peptidases and transporters, a property often leveraged in drug design and peptide-based therapeutics. In some aspects, the azole tripeptide can include each peptide having a S stereocenter. In some aspects, the azole tripeptide includes one, two, or three S stereocenters.

Tripeptides containing one or more amino acid residues with R stereochemistry at the α-carbon (the D-form for most standard amino acids) exhibit altered structural and biological behavior compared to their all-S (L-form) counterparts. The introduction of R-configured residues disrupts the typical right-handed secondary structures favored by L-amino acids, often leading to local distortions in backbone conformation, changes in dihedral angles, and reduced propensity for α-helix or β-sheet formation. This stereochemical inversion can significantly affect intramolecular hydrogen bonding patterns and side chain orientations, thereby influencing the overall folding, solubility, and stability of the tripeptide. Biologically, R-stereochemistry may reduce recognition by peptidases, enhancing resistance to enzymatic degradation, but can also impair binding to native receptors or transporters that are stereoselective for L-forms. As a result, D-amino acid incorporation is used strategically in peptide drug design to modulate pharmacokinetics or bioactivity, albeit with careful consideration of its impact on structure-function relationships. In some aspects, the azole tripeptide can include each peptide having a R stereocenter. In some aspects, the azole tripeptide includes one, two, or three R stereocenters.

Tripeptides containing a mixture of S and R stereochemistry at their α-carbon centers exhibit unique conformational and functional properties distinct from homochiral sequences. The combination of L-(S) and D-(R) amino acids introduces conformational asymmetry, which can disrupt regular secondary structures such as α-helices and β-sheets and instead promote turns, loops, or other noncanonical motifs. This stereochemical diversity influences backbone torsion angles and side chain positioning, often leading to increased structural rigidity or resistance to enzymatic degradation. Mixed S/R stereochemistry can also modulate receptor binding affinity and selectivity, potentially enhancing or inhibiting bioactivity depending on the spatial requirements of the biological target. In peptide drug design, such heterochiral tripeptides are employed to fine-tune pharmacokinetic properties, reduce immunogenicity, or introduce conformational constraints that improve stability and specificity. In some aspects, the azole tripeptide can include at least one peptide having a S stereocenter and at least one peptide having a R stereocenter. In some aspects, the azole tripeptide includes one S stereocenter and two R stereocenters, or vice versa. The azole tripeptide can be R-S-R, S-R-S, R-R-S, S-S-R, or other combinations.

Previously, FDA approved vasodilators typically achieve +115% diameter increases after 10 minutes on the chorioallantoic membrane assay (CAM), but then recede to resting levels thereafter. These phenotypic outcomes were recapitulated in 25% of embryos administered a 100-fold lethal dose of hypoxia-inducing COCl₂ (1 M; 2.4 g/kg) on Day 6, demonstrating the capacity of Compound 1 to rescue embryos in end-stage coronary heart disease. Further, the tripeptide Compound 1 did not induce aberrant angiogenesis or vascular remodeling, as determined by vessel density/branching mapping, and did not weaken the integrity of vessel cell walls, as determined by a Miles assay. Mortality rates were not statistically significant, and no indications of hazardous tumor growth or teratogenic effects were observed after single (Day 6) dosing at 40 μM. The assay was validated against recombinant human VEGF165 (proangiogenic), and NLLMAAS peptide (antiangiogenic). Therefore, Compound 1 and derivatives thereof can be used as vasodilators as described in more detail herein.

Compound 1 and its derivatives can be administered to obtain extended activity without risk of vascular remodeling, cell proliferation or migration. Therefore, Compound 1 and its derivatives can be used for assays to determine: (1) screen for molecules that display the vasodilation phenotype that can be used for treating angina and coronary heart disease, and (2) screen identified molecules that are vasodilators to determine therapeutic potential to treat ischemic disorders, such as peripheral artery disease, ischemic bowel disorder, vascular dementia, and COVID-19.

Compound 1 can be used as a lead scaffold for the development of azole-containing vasodilators with a sustained duration of action for the treatment of systemic arterial pulmonary disorder (SAP). These vasodilators can be used in the treatment of congenital heart disease (CHD), and also in other ischemic conditions, such as peripheral artery disease and ischemic bowel disorder. Additionally, Compound 1 and its derivative can be used in the treatment of neurodegenerative diseases, such as vascular dementia. Also, Compound 1 and its derivatives can be used to treat pulmonary complications or complications arising from COVID-19.

In some embodiments, the vasodilating compound can be an azole tripeptide and have a structure of Formula 1, Formula 1A, or Formula 1B as provided herein, or derivative thereof, prodrug thereof, salt thereof, stereoisomer thereof, or having any chirality at any chiral center, or tautomer, polymorph, solvate, or combination thereof, as presented herein.

In some embodiments, the azole tripeptide compound includes a structure of Formula 1, Formula 1A, or Formula 1B;

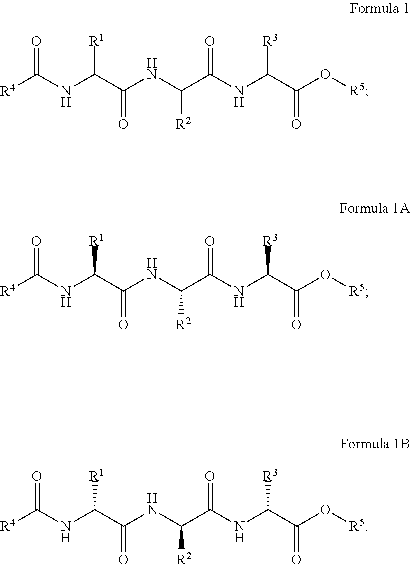

In the formulae, R¹, R², and R³ each independently include a substituent having an azole group that is substituted or unsubstituted with an R group; and R⁴ and R⁵ are each independently end cap moieties. When substituted, the substituent is a chemical moiety, and not a solitary hydrogen.

In some embodiments, the R, R¹, R², R³, R⁴, and R⁵ each independently include hydrogen or a substituent moiety selected from halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, hetero-polyaromatics, substituted hetero-polyaromatics, amines, primary amines, secondary amines, tertiary amines, aliphatic amines, carbonyls, carboxyls, amides, esters, phosphates, alkyl phosphates, phosphonate, alkyl phosphonate, carbamates, alkyl carbamates, amino alkyl carbamates, amino acid carbamates, amino acids, any aryl or cyclo with or without hetero atoms, each being substituted or unsubstituted, or combinations thereof. Each of R¹, R², and R³ include at least one azole group with or without a substituent.

In some embodiments, the R, R¹, R², R³, R⁴, and R⁵ each independently include an alkyl, alkenyl, alkynyl, cycloalkyl, cycloalkenyl, cycloalkynyl, aryl, polyaryl, hetroaryl, polyhetroaryl, alkaryl, aralkyl, halo, halo-substituted alkyl, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof. Each of R¹, R², and R³ include at least one azole group with or without a substituent.

In some embodiments, the R, R¹, R², R³, R⁴, and R⁵ each independently can include any one or more of the substituents selected from the group of, C₁-C₂₄alkyl, C₂-C₂₄alkenyl, C₂-C₂₄alkynyl, C₃-C₂₄cycloalkyl, C₄-C₂₄cycloalkenyl, C₅-C₂₄cycloalkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄alkyl)), di-(C₁-C₂₄alkyl)-substituted carbamoyl (—(CO)—N(C₁-C₂₄alkyl)₂), mono-substituted arylcarbamoyl (—(CO)—NH-aryl), di-substituted arylcarbamoyl (—(CO)—NH-aryl)₂, thiocarbamoyl (—(CS)—NH₂), mono-(C₁-C₂₄ alkyl)-substituted thiocarbamoyl (—(CS)—NH(C₁-C₂₄ alkyl)), di-(C₁-C₂₄ alkyl)-substituted thiocarbamoyl (—(CS)—N(C₁-C₂₄alkyl)₂), mono-substituted arylthiocarbamoyl (—(CS)—NH-aryl), di-substituted arylthiocarbamoyl (—(CS)—NH-aryl)₂, carbamido (NH—(CO)—NH₂), mono-(C₁-C₂₄alkyl)-substituted carbamido (—NH—(CO)—NH(C₁-C₂₄alkyl)), di-(C₁-C₂₄ alkyl)-substituted carbamido (—NH(CO)—N(C₁-C₂₄ alkyl)₂), mono-substituted aryl carbamido (—NH—(CO)—NH-aryl), di-substituted aryl carbamido (—NH—(CO)—N-(aryl)₂), carbamate (—O—(CO)—NH—), alkyl carbamate (—O—(CO)—NH-alkyl), cyano (—C≡N), isocyano (—N⁺≡C⁻), cyanato (—O—C≡N), isocyanato (—O—N⁺≡C⁻), thiocyanato (—S—C≡N), isothiocyanato (—S—N⁺≡C⁻), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₆-C₂₀ aryl)-substituted amino, C₂-C₂₄ alkylamido (—NH(CO)-alkyl), C₅-C₂₀ arylamido (—NH—(CO)-aryl), imino (—CR═NH where R is hydrogen, C₁-C₂₄alkyl, C₅-C₂₀ aryl, C₆-C₂₄ alkaryl, C₆-C₂₄ aralkyl, etc.), alkylimino (CR═N(alkyl), where R=hydrogen, C₁-C₂₄alkyl, aryl, alkaryl, aralkyl, etc.), arylimino (CR═N(aryl), where R=hydrogen, alkyl, aryl, alkaryl, etc.), nitro (—NO₂), nitroso (—NO), sulfonic acid (—SO₂—OH), sulfonato (—SO₂—O⁻), C₁-C₂₄alkylsulfanyl (—S-alkyl; also termed “alkylthio”), C₅-C₂₀ arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O—)), phospho (—PO₂), phosphino (—PH₂), phosphate, sulphate, any with or without hetero atoms (e.g., N, O, P, S, or other) where the hetero atoms can be substituted (e.g., hetero atom substituted for carbon in chain or ring) for the carbons or in addition thereto (e.g., hetero atom added to carbon chain or ring) swapped, any including straight chains, any including branches, and any inducing rings, derivatives thereof, and combinations thereof. Any group can be substituted with halo atoms. Each of R¹, R², and R³ include at least one azole group with or without a substituent.

In some embodiments, the Formula 1 is defined by R⁴ and R⁵ independently including an alkyl, alkenyl, alkynyl, cycloalkyl, cycloalkenyl, cycloalkynyl, aryl, polyaryl, hetroaryl, polyhetroaryl, alkaryl, aralkyl, halo, halo-alkyl, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof.

In certain embodiments, the N-terminus of the polypeptide may be optionally capped with a chemically stable protecting group or functional moiety to enhance pharmacokinetic properties, resist enzymatic degradation, or modulate bioactivity. Suitable N-terminal capping groups include substituted or unsubstituted alkyl groups such as methyl, ethyl, n-propyl, isopropyl, n-butyl, sec-butyl, tert-butyl, pentyl, or hexyl; cycloalkyl groups such as cyclopropyl, cyclobutyl, cyclopentyl, or cyclohexyl; and aryl or arylalkyl groups including phenyl, benzyl, tolyl, xylyl, anisyl, and naphthyl. Additional suitable capping groups include alkoxy groups such as methoxy, ethoxy, propoxy, isopropoxy, and tert-butoxy; aryloxy groups such as phenoxy, benzoxy, 4-methoxyphenyl, and 4-nitrophenyl; acyl groups such as acetyl, propionyl, butyryl, isobutyryl, pivaloyl, benzoyl, and trifluoroacetyl; and sulfonyl groups such as methanesulfonyl, trifluoromethanesulfonyl, benzenesulfonyl, tosyl, brosyl, and nosyl. Further suitable N-terminal modifiers include silyl groups such as trimethylsilyl, triethylsilyl, tert-butyldimethylsilyl, and triisopropylsilyl; heterocyclic moieties including imidazolyl, triazolyl, tetrazolyl, morpholinyl, piperidinyl, piperazinyl, and pyridyl; alkylated amines such as dimethylamino, diethylamino, and morpholino; and carbonates or carbamates including methyl carbonate, ethyl carbonate, phenyl carbonate, methyl carbamate, ethyl carbamate, benzyl carbamate, and tert-butyl carbamate.

In additional embodiments, the C-terminus of the polypeptide may be capped through esterification or amidation to enhance chemical stability, reduce hydrolysis, or alter bioavailability. Suitable ester capping groups include alkyl esters such as methyl ester, ethyl ester, n-propyl ester, isopropyl ester, tert-butyl ester, and pentyl ester, as well as aryl esters such as benzyl ester and phenyl ester. Suitable C-terminal amide capping groups include alkyl amides such as methylamide, ethylamide, isopropylamide, and tert-butylamide; aryl amides such as benzylamide and anilide; and heterocyclic amides including morpholineamide, piperidineamide, and pyrrolidineamide. Additional terminal modifying groups may include ureas and thioureas bearing alkyl or aryl substituents; sulfonamides such as methanesulfonamide, trifluoromethanesulfonamide, and benzenesulfonamide; and silyl-derived groups such as trimethylsilyl esters and tert-butyldimethylsilyl amides. In certain embodiments, salts, solvates, or other derivatives of the aforementioned groups may also be used to cap the C-terminus of the polypeptide.

In some embodiments, the vasodilator azole tripeptide compound has the structure of Formula 2, Formula 2A, or Formula 2B, or derivative thereof, prodrug thereof, salt thereof, stereoisomer thereof, or having any chirality at any chiral center, or tautomer, polymorph, solvate, or combination thereof,

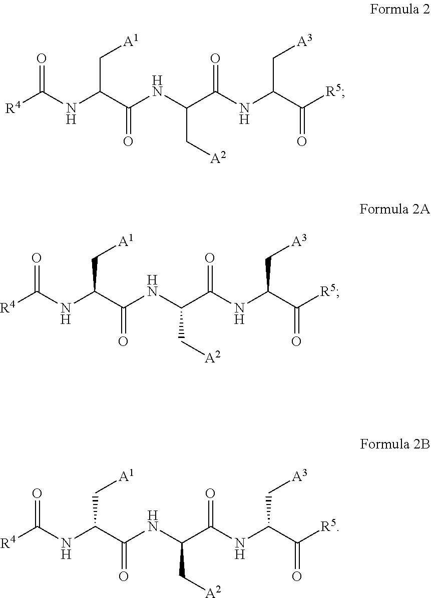

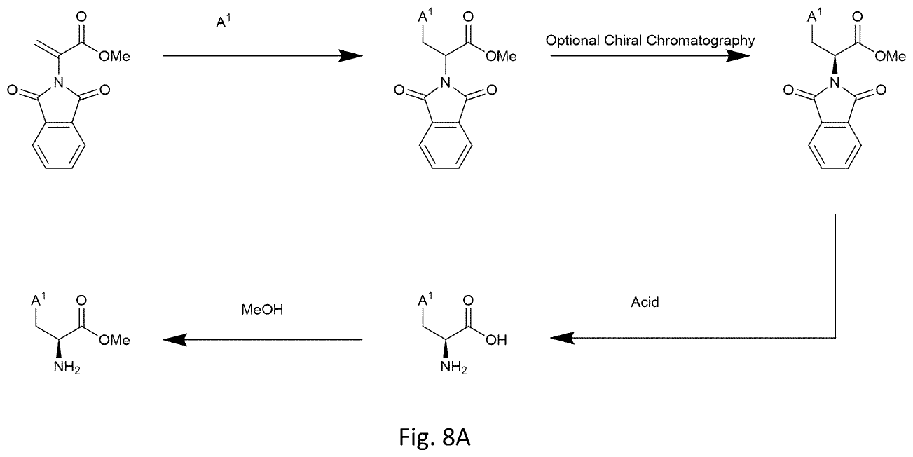

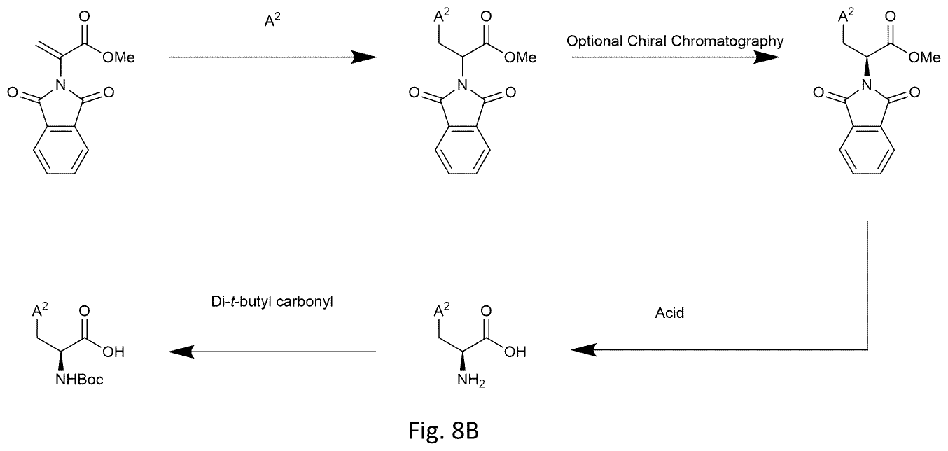

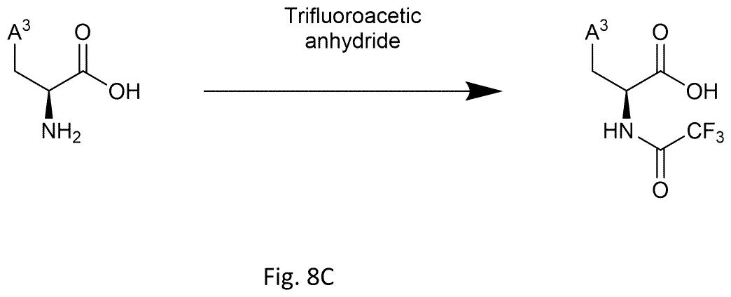

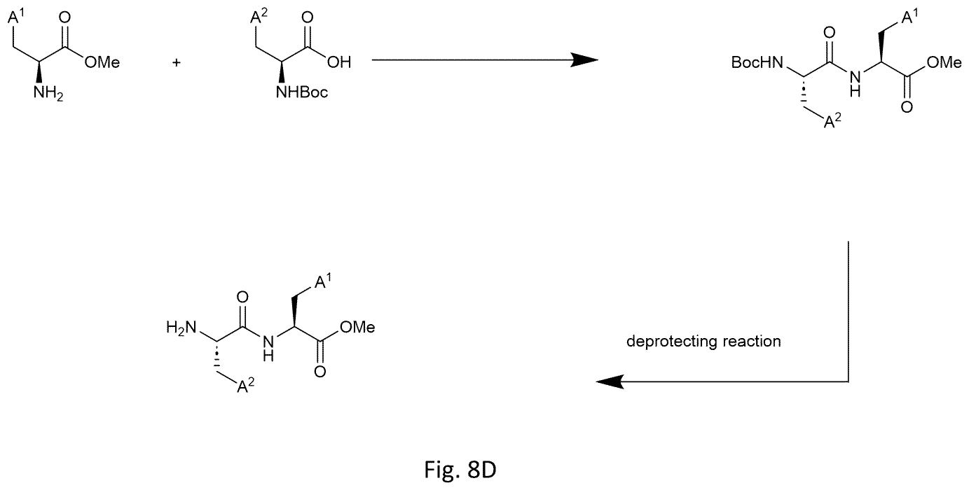

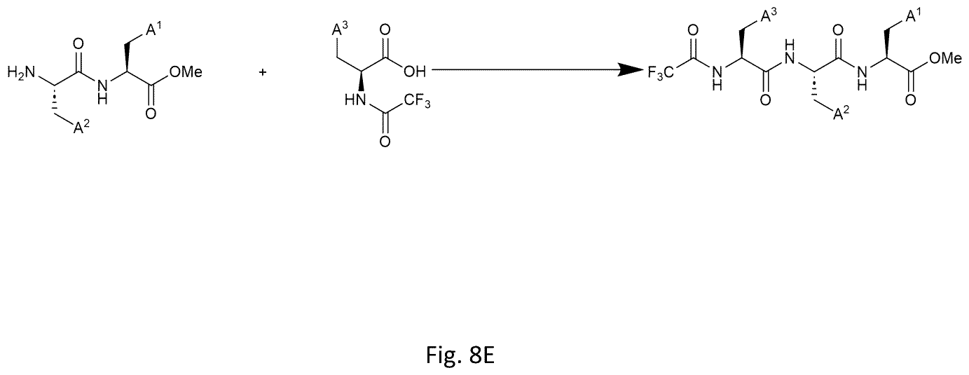

In the formulae, the A¹, A², and A³ can independently include an azole group, and be substituted or unsubstituted. That is each substituent can include at least part that is an azole group, whether the azole is linked to the backbone or whether on another portion of the substituent. Also, any of the azole groups may be substituted or unsubstituted with an R group or adjacent R groups may form a cycle (e.g., cycloalkyl, aryl). The substitution can be at any available carbon atom. In relation to FIGS. 8A-8D, the A¹ and the A³ can be swapped.

As used herein, the term “azole” includes monocyclic or fused bicyclic heteroaryl groups having 5 to 10 ring atoms, containing one to four heteroatoms independently selected from nitrogen, oxygen, and sulfur, and includes substituted or unsubstituted forms of azole, such as pyrrole, imidazole, pyrazole, triazole (1,2,3- and 1,2,4-isomers), tetrazole, oxazole, isoxazole, thiazole, isothiazole, and benzofused analogs such as benzoxazole, benzimidazole, benzothiazole, indole, indazole, carbazole, isoindole, and benzotriazole as well as others.

In some embodiments, the vasodilator azole tripeptide compound has the structure of Formula 3, Formula 3A, or Formula 3B, or derivative thereof, prodrug thereof, salt thereof, stereoisomer thereof, or having any chirality at any chiral center, or tautomer, polymorph, solvate, or combination thereof,

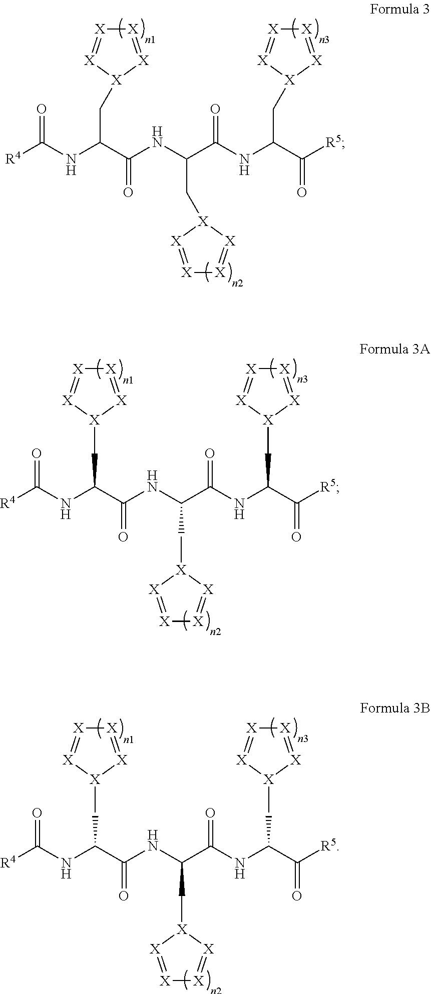

In the formulae, each X is independently carbon, nitrogen, oxygen, and sulfur, so long as one X is carbon and one X is nitrogen, and wherein each X that is carbon is substituted or unsubstituted with an R group, wherein adjacent R groups may form a cycle (e.g., cycloalkyl, aryl). The n1, n2, and n3 are each independently an integer, such as 1, 2, 3, 4, or 5. The X attached to the backbone can be a carbon or a nitrogen, preferably a nitrogen.

In some embodiments, the vasodilator compound has the structure of Formula 4, Formula 4A, or Formula 4B, or derivative thereof, prodrug thereof, salt thereof, or stereoisomer thereof, or having any chirality at any chiral center, or tautomer, polymorph, solvate, or combination thereof,

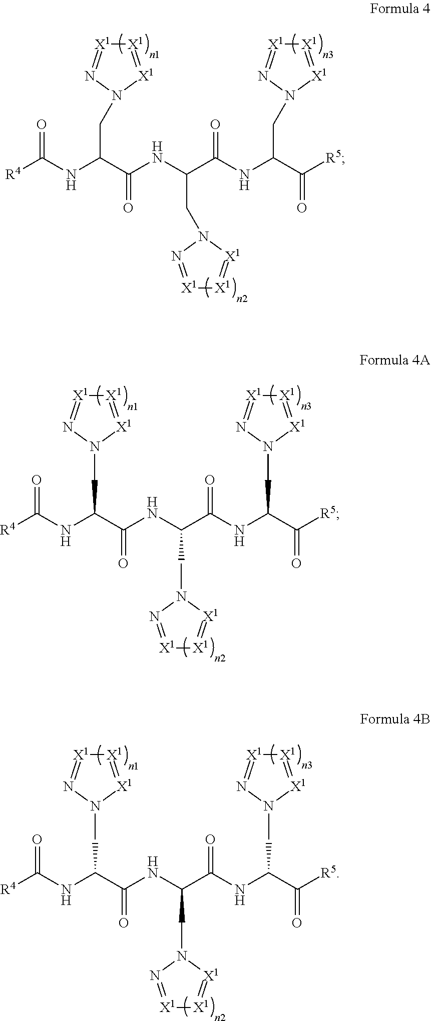

In Formula 4, 4A, and 4B, each X¹ is independently carbon or nitrogen, so long as at least one X¹ is carbon, and wherein each X¹ that is carbon is substituted or unsubstituted with an R group, wherein adjacent R groups may form a cycle (e.g., cycloalkyl, aryl). The n1, n2, and n3 are each independently an integer, such as 1, 2, 3, 4, or 5.

In some embodiments, the vasodilator azole tripeptide compound has the structure of Formulae 5, 5A, or 5B, or derivative thereof, prodrug thereof, salt thereof, or stereoisomer thereof, or having any chirality at any chiral center, or tautomer, polymorph, solvate, or combination thereof,

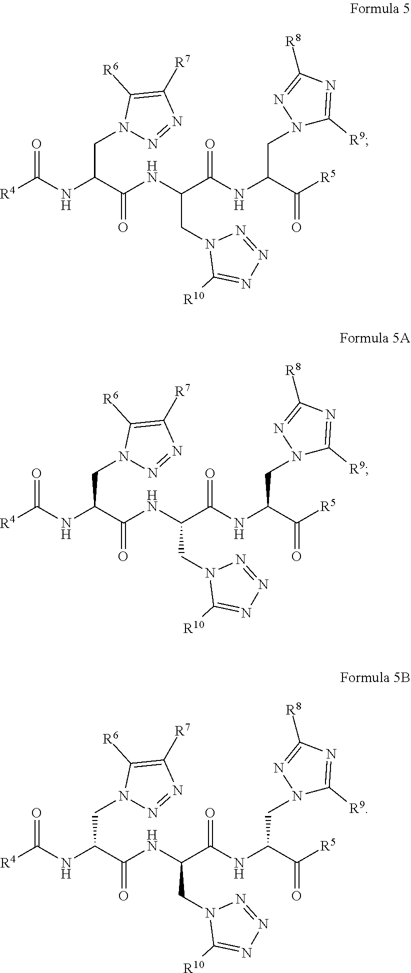

In the formulae, the R⁶ and R⁷ can independently be hydrogen or an R group substituent as defined herein, or may form a cycle (e.g., cycloalkyl, aryl), and R⁸, R⁹, and R¹⁰ can independently be hydrogen or an R group substituent as defined herein.

In some embodiments, R⁶ and R⁷ as well as R⁸, R⁹, and R¹⁰ are independently a hydrogen or a substituent, wherein the substituent includes alkyl, alkenyl, alkynyl, cycloalkyl, cycloalkenyl, cycloalkynyl, aryl, polyaryl, hetroaryl, polyhetroaryl, alkaryl, aralkyl, halo, halo-substituted alkyl, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof.

In some embodiments, R⁶, R⁷, R⁸, R⁹, and R¹⁰ are each hydrogen or a substituent selected from the group of C₁-C₂₄alkyl, C₂-C₂₄alkenyl, C₂-C₂₄alkynyl, C₃-C₂₄cycloalkyl, C₄-C₂₄ cycloalkenyl, C₅-C₂₄ cycloalkynyl, C₅-C₂₀ aryl, C₆-C₂₄ alkaryl, C₆-C₂₄ aralkyl, halo, hydroxyl, sulfhydryl, C₁-C₂₄alkoxy, C₂-C₂₄alkenyloxy, C₂-C₂₄ alkynyloxy, C₅-C₂₀ aryloxy, acyl (including C₂-C₂₄alkylcarbonyl (—CO-alkyl) and C₆-C₂₀ arylcarbonyl (CO-aryl)), acyloxy (O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄alkyl)₂), mono-substituted arylcarbamoyl (—(CO)—NH-aryl), di-substituted arylcarbamoyl (—(CO)—NH-aryl)₂, thiocarbamoyl (—(CS)—NH₂), mono-(C₁-C₂₄ alkyl)-substituted thiocarbamoyl (—(CS)—NH(C₁-C₂₄ alkyl)), di-(C₁-C₂₄ alkyl)-substituted thiocarbamoyl (—(CS)—N(C₁-C₂₄ alkyl)₂), mono-substituted arylthiocarbamoyl (—(CS)—NH-aryl), di-substituted arylthiocarbamoyl (—(CS)NH-aryl)₂, carbamido (NH—(CO)—NH₂), mono-(C₁-C₂₄alkyl)-substituted carbamido (—NH—(CO)—NH(C₁-C₂₄ alkyl)), di-(C₁-C₂₄ alkyl)-substituted carbamido (—NH(CO)—N(C₁-C₂₄alkyl)₂), mono-substituted aryl carbamido (—NH—(CO)—NH-aryl), di-substituted aryl carbamido (—NH—(CO)N-(aryl)₂), carbamate (—O—(CO)—NH—), alkyl carbamate (—O—(CO)—NH-alkyl), cyano (—C≡N), isocyano (—N⁺≡C⁻), cyanato (—O—C≡N), isocyanato (—O—N⁺≡C⁻), thiocyanato (—S—C≡N), isothiocyanato (—S—N⁺≡C⁻), azido (—N═N⁺≡N⁻), formyl (—(CO)—H), thioformyl (—(CS)—H), amino (—NH₂), mono- and di-(C₁-C₂₄alkyl)-substituted amino, mono- and di-(C₆-C₂₀ aryl)-substituted amino, C₂-C₂₄ alkylamido (—NH(CO)-alkyl), C₅-C₂₀ arylamido (—NH(CO)-aryl), imino (—CR═NH where R is hydrogen, C₁-C₂₄alkyl, C₅-C₂₀ aryl, C₆-C₂₄ alkaryl, C₆-C₂₄ aralkyl, etc.), alkylimino (CR═N(alkyl), where R=hydrogen, C₁-C₂₄alkyl, aryl, alkaryl, aralkyl, etc.), arylimino (CR═N(aryl), where R=hydrogen, alkyl, aryl, alkaryl, etc.), nitro (—NO₂), nitroso (—NO), sulfonic acid (—SO₂—OH), sulfonato (—SO₂—O⁻), C₁-C₂₄ alkylsulfanyl (—S-alkyl; also termed “alkylthio”), C₅-C₂₀ arylsulfanyl (—S-aryl; also termed “arylthio”), C₁-C₂₄alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (SO₂-aryl), phosphono (—P(O)(OH)₂), phosphonato (—P(O)(O⁻)₂), phosphinato (—P(O)(O⁻)), phospho (—PO₂), phosphino (—PH₂), phosphate, sulphate, any with or without hetero atoms (e.g., N, O, P, S, or other) where the hetero atoms can be substituted (e.g., hetero atom substituted for carbon in chain or ring) for the carbons or in addition thereto (e.g., hetero atom added to carbon chain or ring) swapped, any including straight chains, any including branches, and any inducing rings, derivatives thereof, and combinations thereof. Any group can be substituted with halo atoms.

In some embodiments, the alkyl, alkenyl, and alkynyl can be C₁-C₁₂ alkyl, C₂-C₁₂ alkenyl, C₂-C₁₂alkynyl, or C₁-C₆alkyl, C₂-C₆alkenyl, C₂-C₆alkynyl, or C₁-C₄alkyl, C₂-C₄alkenyl, C₂-C₄alkynyl.

In some embodiments, the cycle formed by adjacent R groups, such as R⁶ and R⁷, can include a cycloalkyl, which is substituted or unsubstituted. As used herein, the term “cycloalkyl” refers to a saturated or partially saturated monocyclic, bicyclic, or tricyclic hydrocarbon ring system having 3 to 12 carbon atoms. Representative examples of cycloalkyl groups include, but are not limited to, cyclopropyl, cyclobutyl, cyclopentyl, cyclohexyl, cycloheptyl, cyclooctyl, cyclononyl, cyclodecyl, cycloundecyl, cyclododecyl, bicyclo[1.1.0]butyl, bicyclo[2.2.1]heptyl (norbornyl), bicyclo[2.2.2]octyl, adamantyl, and noradamantyl. The cycloalkyl groups may be optionally substituted with one or more substituents independently selected from alkyl, halo, hydroxy, alkoxy, carboxy, cyano, amino, alkylamino, dialkylamino, nitro, or halogenated alkyl.

In some embodiments, the cycle formed by adjacent R groups, such as R⁶ and R⁷, can include an aryl. As used herein, the term “aryl” refers to a monocyclic or polycyclic aromatic hydrocarbon ring system containing 6 to 14 carbon atoms. Aryl groups include, but are not limited to, phenyl, naphthyl (e.g., 1-naphthyl, 2-naphthyl), anthracenyl, phenanthryl, biphenyl, terphenyl, and fluorenyl. Aryl groups may be optionally substituted with one or more substituents independently selected from halo, hydroxy, amino, nitro, cyano, alkyl, alkenyl, alkynyl, alkoxy, aryloxy, aryl, heteroaryl, acyl, carboxy, carboxyalkyl, carbamoyl, sulfonyl, sulfonamido, halogenated alkyl, or haloalkoxy. The term “aryl” also includes fused aromatic systems such as benzofused aryl groups, for example, indanyl, indenyl, fluorenyl, and other polycyclic systems in which at least one aromatic ring is fused to one or more additional rings, which may be aromatic or non-aromatic.

The aryl group formed by R⁶ and R⁷ may be a hetero aryl. As used herein, the term “heteroaryl” refers to a monocyclic, bicyclic, or tricyclic aromatic ring system containing one to four heteroatoms independently selected from nitrogen (N), oxygen (O), and sulfur (S), where the ring system contains 5 to 14 ring atoms in total. The heteroaryl group may be unsubstituted or optionally substituted with one or more substituents as defined below. Representative examples of heteroaryl groups include, but are not limited to: pyridyl (e.g., 2-pyridyl, 3-pyridyl, 4-pyridyl), pyrimidinyl, pyrazinyl, pyridazinyl, pyrazolyl, imidazolyl, triazolyl (1,2,3- and 1,2,4-isomers), tetrazolyl, oxazolyl, isoxazolyl, thiazolyl, isothiazolyl, furyl, thienyl, pyrrolyl, indolyl, isoindolyl, indazolyl, benzofuranyl, benzothienyl, benzoxazolyl, benzimidazolyl, benzothiazolyl, quinolyl, isoquinolyl, cinnolinyl, quinazolinyl, quinoxalinyl, and phthalazinyl. Heteroaryl groups may be optionally substituted with one or more substituents independently selected from alkyl, alkenyl, alkynyl, halo, hydroxy, cyano, nitro, amino, alkylamino, dialkylamino, acyl, carboxy, alkoxy, aryloxy, aryl, heteroaryl, haloalkyl, haloalkoxy, sulfonyl, sulfonamido, and carbamoyl.

In some embodiments, the R group substituents can include halo (fluoro, chloro, bromo, iodo), hydroxyl, cyano, nitro, amino (—NH₂), alkylamino, dialkylamino, carboxy (—COOH), alkoxy (—OR), aryloxy, acyl (—C(O)R), alkylcarbonyl, alkoxycarbonyl, carbamoyl (—CONH₂), alkylcarbamoyl, dialkylcarbamoyl, sulfonyl (—SO₂R), sulfonamido (—SO₂NH₂), thiol (—SH), thioether (—SR), alkyl, alkenyl, alkynyl, haloalkyl, haloalkoxy, cycloalkyl, and heterocyclyl. Unless otherwise indicated, the alkyl, alkenyl, and alkynyl refer to straight, branched, or cyclic chains containing 1 to 10 carbon atoms. Substituents may be mono- or polysubstituted and may be independently selected at each substitution position.

In some embodiments, the azole groups can be part of fluconazole, clotrimazole, rizatriptan, alprazolam, losartan, valsartan, letrozole, vorozole, or other.

In some embodiments, the azole tripeptides can be selected from any of the following:

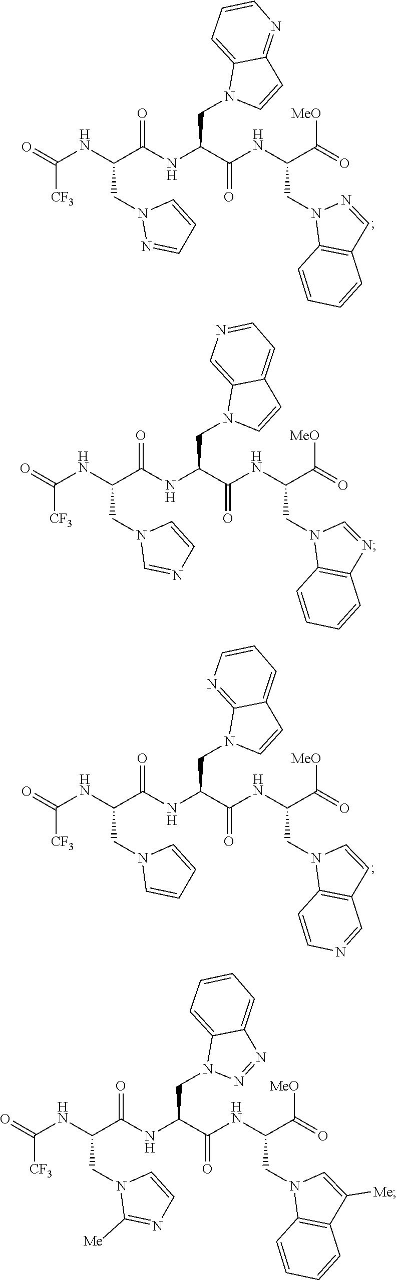 [SMILES: COC(=O)[C@H](Cn1ncc2ccccc21)NC(=O)[C@H](Cn1ccc2ncccc21)NC(=O)[C@H](Cn1cccn1)NC(=O)C(F)(F)F ; COC(=O)[C@H](Cn1cnc2ccccc21)NC(=O)[C@H](Cn1ccc2ccncc21)NC(=O)[C@H](Cn1ccnc1)NC(=O)C(F)(F)F ; COC(=O)[C@H](Cn1ccc2cnccc21)NC(=O)[C@H](Cn1ccc2cccnc21)NC(=O)[C@H](Cn1cccc1)NC(=O)C(F)(F)F ; COC(=O)[C@H](Cn1cc(C)c2ccccc21)NC(=O)[C@H](Cn1nnc2ccccc21)NC(=O)[C@H](Cn1ccnc1C)NC(=O)C(F)(F)F]

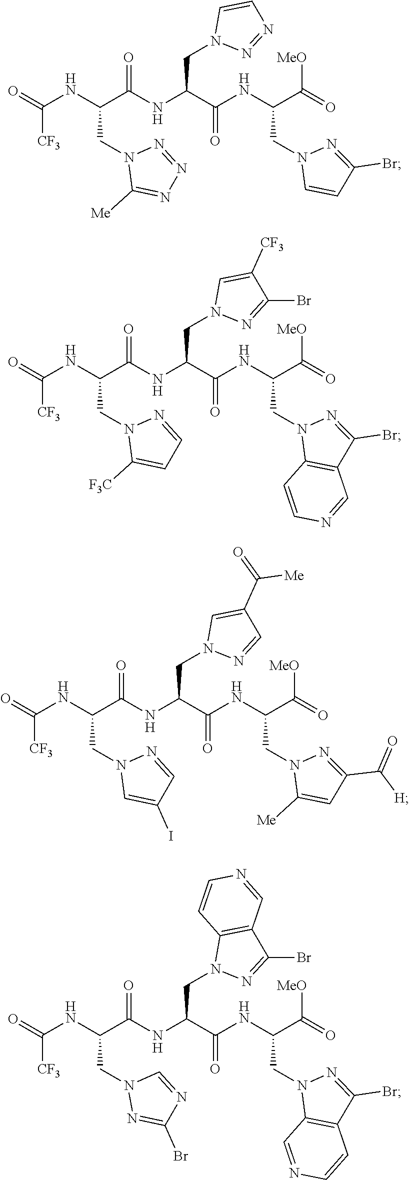

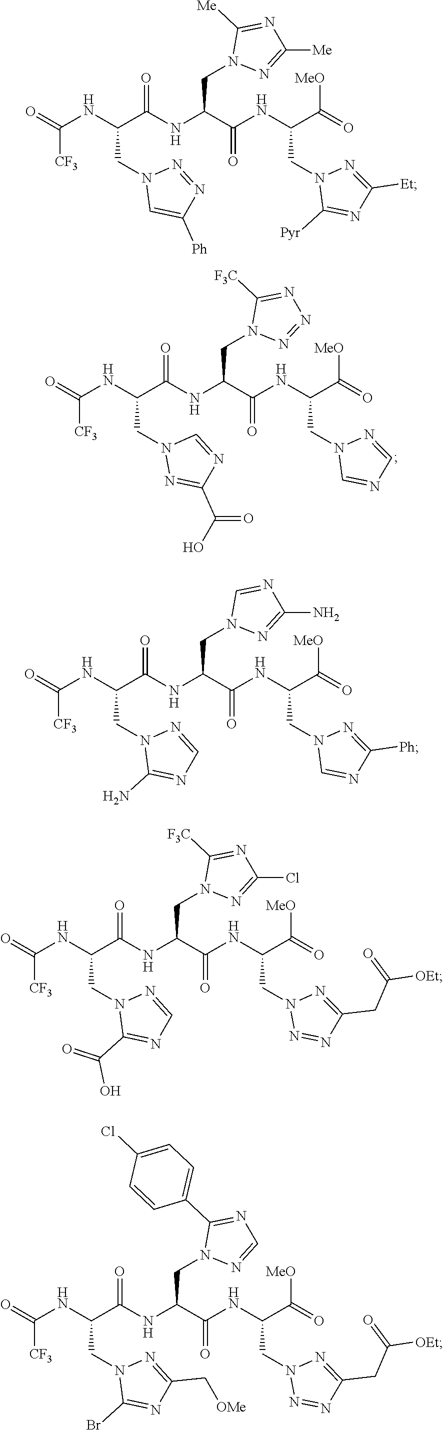

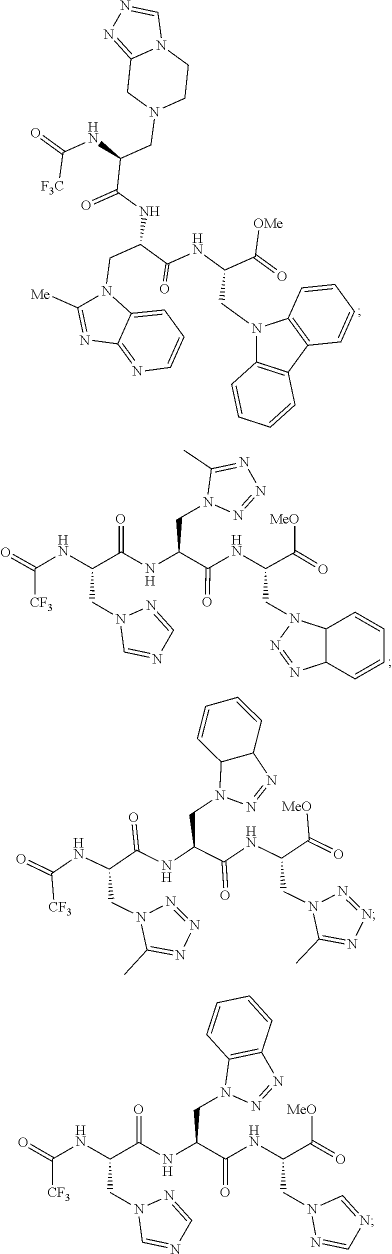

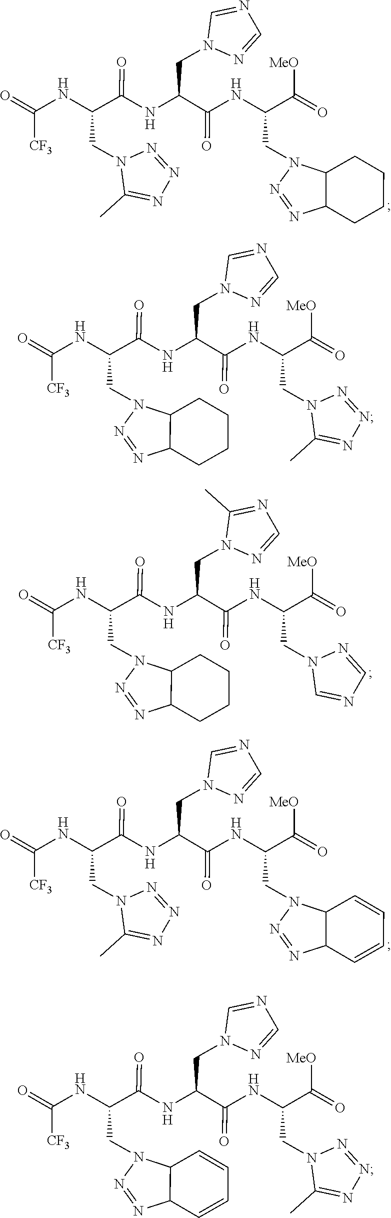

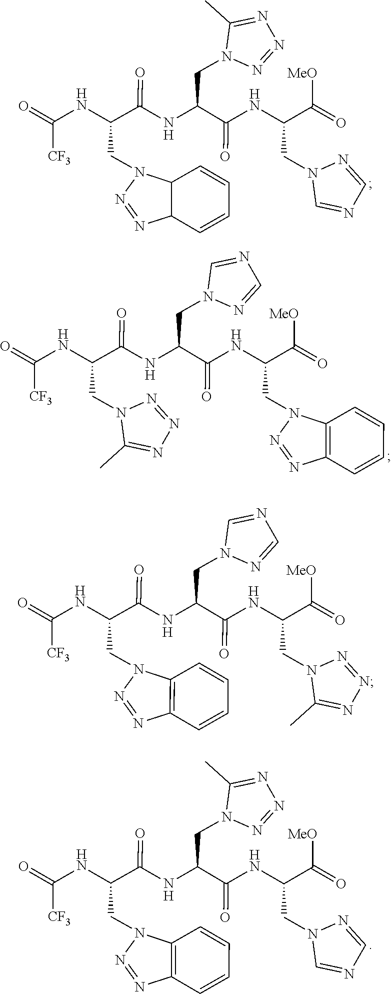

In the compounds illustrated herein, it should be recognized that any of the peptide chiral centers can be S (e.g., L) or R (e.g., D) stereocenters. Accordingly, the chiral centers may be all S, all R, or a combination of S and R stereocenters.

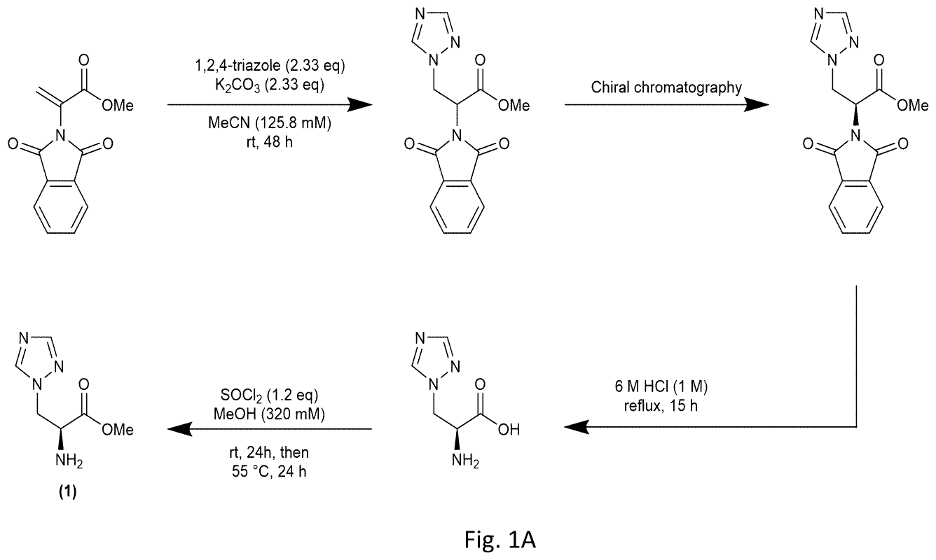

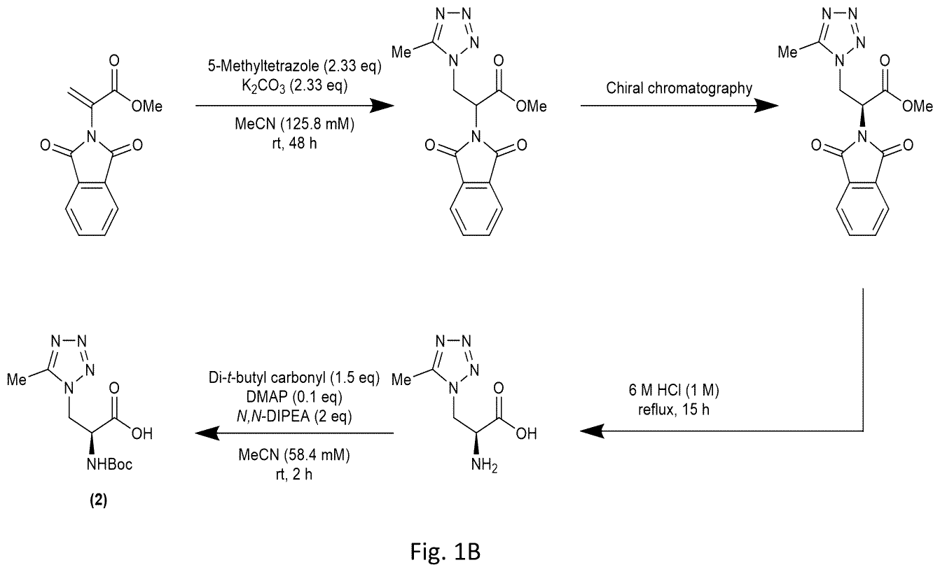

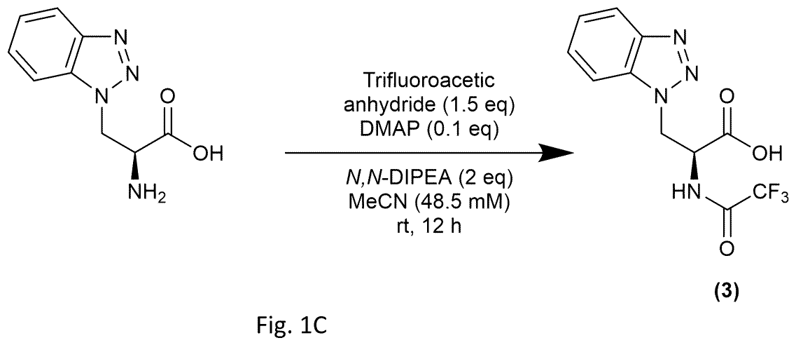

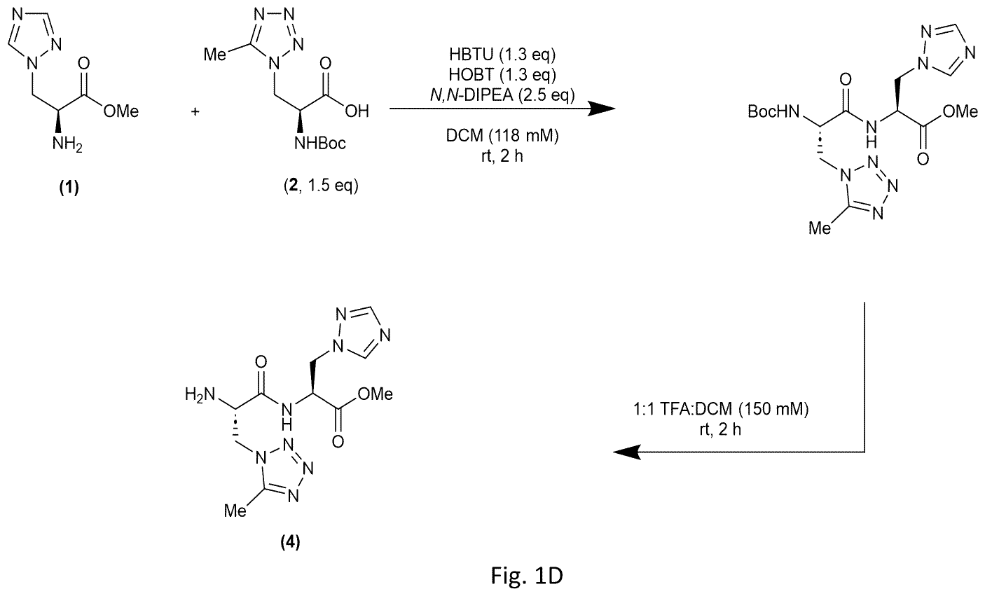

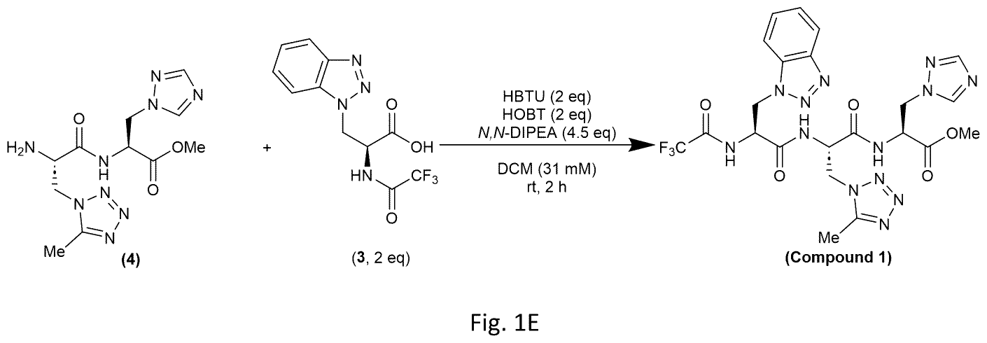

In some embodiments, the synthesis of the vasodilator compound can include the reaction schemes in FIGS. 1A-1E and 8A-8E.

In some embodiments, the compounds described herein can be in the form of a pharmaceutically acceptable salt. As used herein, the term “pharmaceutically acceptable salt” refers to those salts which are suitable to use within mammals and do not tend to be toxic. Pharmaceutically acceptable salts are formed using inorganic and organic acids and bases. Examples of pharmaceutically acceptable salts are salts of an amino group formed with inorganic acids such as hydrochloric acid, hydrobromic acid, phosphoric acid, sulfuric acid and perchloric acid, or with organic acids such as tartaric acid, acetic acid, oxalic acid, maleic acid, citric acid, succinic acid or malonic acid, terephthalic acid. Other pharmaceutically acceptable salts include adipate, ascorbate, aspartate, benzoate, bisulfate, borate, butyrate, valerate, camphorate, camphorsulfonate, cyclopentanepropionate, formate, citrate, oxalate, pivalate, succinate, tartrate, fumarate, gluconate, hemisulfate, heptanoate, hexanoate, hydroiodide, 2-hydroxy-ethanesulfonate, lactate, laurate, lauryl sulfate, maleate, malonate, methanesulfonate, 2-naphthalenesulfonate, nicotinate, nitrate, oleate, palmitate, stearate, undecanoate, alginate, 3-phenylpropionate, phosphate, sulfate, thiocyanate, p-toluenesulfonate, benzenesulfonate, persulfate, ethanesulfonate, dodecylsulfate, and the like and mixture salts.

Salts derived from appropriate bases include alkali metal, alkaline earth metal, ammonium and N(Calkyl) salts. Representative alkali or alkaline earth metal salts include Sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, alkyl sulfonate and aryl sulfonate.

In certain embodiments, the compounds described herein may be administered in the form of a prodrug comprising a cleavable prodrug moiety covalently attached to a biologically active drug to improve pharmacokinetic, physicochemical, or targeting properties. Suitable prodrug-forming moieties include ester-forming groups such as aliphatic or aromatic carboxylic acids, including acetic acid, propionic acid, benzoic acid, and pivalic acid; amino acid derivatives such as glycine, alanine, valine, leucine, phenylalanine, and proline; phosphates and phosphonates including monoesters, diesters, and cyclic phosphates; carbamates including methyl carbamate, ethyl carbamate, and tert-butyl carbamate; carbonates including methyl carbonate, ethyl carbonate, and aryl carbonates; sulfonates including methane sulfonate and benzenesulfonate; and prodrug moieties derived from sugars such as glucose, mannose, and galactose. In some embodiments, the prodrug moiety may also comprise biodegradable linkers such as esters, disulfides, hydrazones, or self-immolative spacers that enable site-specific or enzyme-triggered drug release. The prodrug may be hydrolyzed or enzymatically cleaved in vivo to release the parent drug in its active form.

### Pharmaceutical Compositions

In yet another aspect of the present invention, a pharmaceutical composition is provided comprising one or more azole tripeptide compounds as indicated above or a salt thereof, and a pharmaceutically acceptable carrier or diluent.

As used herein, the phrase “pharmaceutically acceptable route of administration” or “route of administration” includes, but is not limited to, oral, sublingual, buccal, nasal, inhalational (pulmonary), rectal, vaginal, topical, transdermal, ocular (ophthalmic), nasal, otic, parenteral, and mucosal routes. In some aspects, parenteral administration of the compounds is provided. The term “parenteral” as used herein includes subcutaneous, intravenous, intramuscular, and the like.

Pharmaceutically acceptable compositions of this invention are orally administered in any orally acceptable dosage form. Exemplary oral dosage forms are capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch.

Pharmaceutically acceptable compositions of this invention comprising the compounds are also administered topically, especially when the target of treatment includes areas or organs readily accessible by topical application, including diseases of the eye, the skin, or the lower intestinal tract. Suitable topical formulations are readily prepared for each of these areas or organs.

For topical applications, provided pharmaceutically acceptable compositions are formulated in a suitable ointment wherein the compound of the invention, optionally with other active components, is suspended or dissolved in one or more carriers. Exemplary carriers for topical administration of compounds of this are mineral oil, propylene glycol, polyoxyethylene and water. Suitable topical carriers include, but are not limited to, mineral oil, sorbitan monostearate, polysorbates, cetyl alcohol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing certain conservants, including benzyl alcohol or other suitable preservatives, and/or other conventional solubilizing or dispersing agents.

The amount of the compounds of the present invention of the present invention that are optionally combined with the carrier of vehicle materials to produce a composition in a single dosage form for treating a subject will vary depending upon the patient treated, the mode of administration. Preferably, provided compositions should be formulated so that a dosage of between 0.01-100 mg/kg body weight/day of the compound can be administered to a patient receiving these compositions.

The compounds of the present invention can also be in micro-encapsulated form with one or more excipients as noted above. The solid dosage forms of tablets, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose, or starch. Such dosage forms also comprise, as is normal practice, additional substances other than inert diluents, such as tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Additionally, muti-layered dosage forms can be used, which can provide for modified and controlled release of the drug from the oral formulation. The dosage form may be configured with pH sensitive moieties or other stimuli sensitive moieties to selectively and controllably release the formulation in the desired location, whether stomach, small intestine or large intestine.

Dosage forms for topical or transdermal administration of a compound of this invention include ointments, pastes, creams, lotions, gels, powders, solutions, sprays, inhalants, or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, a method of dilating a blood vessel can include: providing a compound in accordance with one of the embodiments; and administering the compound to a subject. In some aspects, the subject is in need of vasodilation. In some aspects, the subject has a condition that is treated with the compound, wherein the condition is hypertension, angina, coronary heart disease, heart failure, Raynaud's disease, peripheral artery disease, ischemic bowel disorder, vascular dementia, COVID-19, other ischemic disorders, or combinations thereof.

### Synthesis

The synthesis of Compound 1 leverages novel synthetic chemistry. The chiral amino acids that are used to construct Compound 1 are not commercial and the coupling conditions have not previously been reported. The technical preparation of the tripeptide involves an innovative synthetic technology that is based on the addition of three separate azoles to a common phthalimido-protected dehydroalanine residue, giving three independent amino acids. The phthalimido protecting group allows for the synthesis to obtain the enantiopure amino acids from the racemic mixture by chiral chromatography. The three chiral amino acids can then be coupled together in a defined order to obtain the beta-azole tripeptide using HBTU/HOBT/DIPEA coupling chemistry. This strategy is conducive to analog synthesis with different azoles.

In some embodiments, a method of preparing an azole tripeptide is provided. The reaction can include: coupling an azole molecule with an oxoisoindolinyl acrylate to obtain an azoleoxoisoindolinyl ester (Scheme 1, FIGS. 1A, 8A); reacting the azole oxoisoindolinyl ester with acid to remove the oxoisoindolinyl and obtain a first azole peptide with a first azole group (Scheme 1, FIGS. 1A, 8A); reacting the first azole peptide with methyl alcohol to obtain an azole peptide methyl ester (Scheme 1, FIGS. 1A, 8A); obtaining a second azole peptide with a second azole group, wherein the second azole group is the same or different from the first azole group (Scheme 2, FIGS. 1B, 8B); reacting the second azole peptide with a nitrogen protecting group to obtain an nitrogen protected azole peptide (Scheme 2, FIGS. 1B, 8B); obtaining a third azole peptide with a third azole group (Scheme 3, FIGS. 1C, 8C), wherein the third azole group is the same or different from the first azole group or the second azole group; reacting the third azole peptide with a trihaloacetic anhydride to obtain an azole trifluoromethylamide (Scheme 3, FIGS. 1C, 8C); reacting the azole peptide methyl ester with the nitrogen protected azole peptide to form nitrogen protected azole dipeptide (Scheme 4, FIGS. 1D, 8D); deprotecting the nitrogen protected azole dipeptide to obtain dipeptide with methyl ester (Scheme 4, FIGS. 1D, 8D); and reacting the dipeptide with methyl ester with azole trifluoromethylamide to obtain the Compound 1 (Scheme 5, FIGS. 1E, 8E).

### Synthesis of methyl (S)-2-amino-3-(1H-1,2,4-triazol-1-yl)propanoate (Scheme 1)

Scheme 1 is shown in FIG. 1A, and includes the following protocol. 1000 mg (14.48 mmol) of 1,2,4-triazole and 2001 mg of K₂CO₃ (14.48 mmol, 1 eq) were placed in a 250 mL round-bottom flask equipped with a stir bar. 50 mL of acetonitrile were added, and the reaction mixture was allowed to stir at room temperature for 15 minutes. After this time, 1445 mg (6.29 mmol, 0.43 eq) of methyl 2-(1,3-dioxoisoindolin-2-yl)acrylate was added and the reaction mixture was further stirred for 48 hours. After this time the reaction mixture was filtered, concentrated under vacuum, suspended in 50 mL of water, and extracted three times with 50 mL of EtOAc. The organic fractions were combined, washed with brine, dried with Mg₂SO₄, and concentrated under vacuum. The product was then purified by normal phase flash chromatography using a silica gel column and EtOAc 0-100% on hexanes as mobile phase. Racemic product was obtained as a white solid and the identity confirmed by NMR. Enantiomers were later separated by chiral chromatography using a chiralpak IA 25×250 mm (5 μm pore size) column and 1:1 chloroform:hexanes as mobile phase by injecting 1 mL of a 20 mg/mL solution of racemic compound in mobile phase at a time. The R-enantiomer eluted at approximately 51 minutes and the S-enantiomer eluted at approximately 63 minutes. Stereochemistry was confirmed by polarimetry on globally deprotected amino acid applying the Clough, Lutz, and Jirgensons rule (J. Chem. Educ. 2018, 95, 10, 1872-1874).

Enantiomerically pure material was then suspended in a 6M HCl solution and heated at reflux for 15 hours using a reflux condenser. After this time, the reaction mixture was concentrated under vacuum until approximately 2 mL of mixture remained. Excess precipitate was removed by filtration and the filtrate was purified by reverse phase chromatography using a C₁₈-silica column and acetonitrile 0-85% in water as mobile phase. Globally deprotected amino acid was obtained as a white solid.

Globally deprotected amino acid (5 mg, 0.032 mmol) was later placed in a 1-dram vial equipped with a stir bar and suspended in 0.1 mL of MeOH. 2.97 μL (4.57 mg, 0.038 mmol, 1.2 eq) of SOCl₂ was slowly added and the reaction was stirred at room temperature for 24 h and later at 55° C. until starting material was not detected by TLC (ninhydrin stain). The product was later purified by reverse phase chromatography using a C₁₈-silica column and acetonitrile 0-85% in water as mobile phase and then by normal phase chromatography using a silica column and MeOH 0-20% in dichloromethane as mobile phase. The product was obtained as a white solid.

### Synthesis of (S)-2-[(t-butoxycarbonyl)amino]-3-(5-methyl-1H-tetrazol-1-yl)propanoic acid (Scheme 2)

Scheme 2 is shown in FIG. 1B, and includes the following protocol. 1217 mg (14.48 mmol) of 5-methyl-tetrazole and 2001 mg of K₂CO₃ (14.48 mmol, 1 eq) were placed in a 250 mL round-bottom flask equipped with a stir bar. 50 mL of acetonitrile was added, and the reaction mixture was allowed to stir at room temperature for 15 minutes. After this time, 1445 mg (6.29 mmol, 0.43 eq) of methyl 2-(1,3-dioxoisoindolin-2-yl)acrylate was added and the reaction mixture was further stirred for 48 hours. After this time the reaction mixture was filtered, concentrated under vacuum, suspended in 50 mL of water and extracted three times with 50 mL of EtOAc. The organic fractions were combined, washed with brine, dried with Mg₂SO₄ and concentrated under vacuum. The product was then purified by normal phase flash chromatography using a silica gel column and EtOAc 0-100% on hexanes as mobile phase. Racemic product was obtained as a white solid and the identity confirmed by NM R. Enantiomers were later separated by chiral chromatography using a chiralpak IA 25×250 mm (5 μm pore size) column and 1:1 chloroform:hexanes as mobile phase by injecting 1 mL of a 20 mg/mL of racemic compound in mobile phase at a time. The R-enantiomer eluted at approximately 70 minutes and the S-enantiomer eluted at approximately 115 minutes. Stereochemistry was confirmed by polarimetry on globally deprotected amino acid applying the Clough, Lutz, and Jirgensons rule.

Enantiomerically pure material was then suspended in a 6M HCl solution and heated at reflux for 15 hours using a reflux condenser. After this time, the reaction mixture was concentrated under vacuum until approximately 2 mL of mixture remained. Excess precipitate was removed by filtration and the filtrate was purified by reverse phase chromatography using a C₁₈-silica column and acetonitrile 0-85% in water as mobile phase. Globally deprotected amino acid was obtained as a white solid.

Globally deprotected amino acid (50 mg, 0.292 mmol), and 3.6 mg (0.0292 mmol, 0.1 eq) of DMAP were later placed in a 50 mL round bottom flask equipped with a stir bar and suspended in 5 mL of acetonitrile. 100.6 μL (95.6 mg, 0.438 mmol, 1.5 eq) of Di-t-butyl decarbonate and 101.7 μL (75.5 mg, 0.584 mmol, 2 eq) of N,N-Diisopropylethylamine was slowly added and the reaction was stirred at room temperature for 2 h. The product was later purified by reverse phase chromatography using a C₁₈-silica column and acetonitrile 0-85% in water as mobile phase. The product was obtained as a white solid.

### Synthesis of (S)-3-(1H-benzo[d][1,2,3]triazol-1-yl)-2-(2,2,2-trifluoroacetamido)propanic acid (Scheme 3)

Scheme 3 is shown in FIG. 1C, and includes the following protocol. Commercially available (S)-2-ammonium-3-(1H-benzo[d][1,2,3]triazol-1-yl)propanoic acid hydrochloride was purified by reverse phase chromatography using a C₁₈-silica column and acetonitrile 0-85% in water as mobile phase. The obtained pure amino acid (100 mg, 0.485 mmol), and 5.9 mg (0.0485 mmol, 0.1 eq) of DMAP were later placed in a 50 mL round bottom flask equipped with a stir bar and suspended in 10 mL of acetonitrile. 101.1 μL (152.8 mg, 0.727 mmol, 1.5 eq) of trifluoroacetic anhydride and 169.0 μL (125.4 mg, 0.970 mmol, 2 eq) of N,N-Diisopropylethylamine were slowly added and the reaction was stirred at room temperature for 12 h. The product was later purified by normal phase chromatography using a silica gel column and methanol 0-10% in dichloromethane as mobile phase. The product was obtained as a white to light yellow solid.

### Synthesis of methyl (S)-2-((S)-2-amino-3-(5-methyl-1H-tetrazol-1-yl)propanamido)-3-(1H-1,2,4-triazol-1-yl)propanoate (Scheme 4)

Scheme 4 is shown in FIG. 1D, and includes the following protocol. 47.8 mg (0.176 mmol) of (S)-2-[(t-butoxycarbonyl)amino]-3-(5-methyl-1H-tetrazol-1-yl)propanoic acid was combined with 20 mg of methyl (S)-2-amino-3-(1H-1,2,4-triazol-1-yl)propanoate (0.118 mmol, 0.67 eq), 30.9 mg of HOBT (0.229 mmol, 1.3 eq), and 86.8 mg (0.229 mmol, 1.3 eq) of HBTU. The mixture was dissolved in 1 mL of dichloromethane with stirring and 76.6 μL of N,N-Diisopropylethylamine (56.9 mg, 0.44 mmol, 2.5 eq) were added. The mixture was allowed to stir at room temperature for 2 h, after which it was concentrated under vacuum, dissolved in 1 mL of methanol, and purified by HPLC using an Alltech Econosil C18 10U 10×250 mm column and acetonitrile 0-95% in water as mobile phase. N-Boc protected dipeptide was obtained as crystalline white solid.

15 mg of N-Boc protected dipeptide were dissolved in 100 μL of a 1:1 TFA:DCM mixture and stirred at room temperature for two hours. The mixture was concentrated under vacuum redissolved in 1 mL of water and purified by HPLC using an Alltech Econosil C18 10U 10×250 mm column and acetonitrile 0-95% in water as mobile phase. Product was obtained as a white solid.

### Synthesis of Compound 1

Scheme 5 is shown in FIG. 1E, and includes the following protocol. 10 mg (0.031 mmol) of dipeptide methyl ester were combined with 18.7 mg of (S)-3-(1H-benzo[d][1,2,3]triazol-1-yl)-2-(2,2,2-trifluoroacetamido)propanic acid (0.062 mmol, 2 eq), 8.4 mg of HOBT (0.062 mmol, 2 eq), and 23.5 mg (0.062 mmol, 2 eq) of HBTU. The mixture was dissolved in 1 mL of dichloromethane with stirring and 24.3 μL of N,N-Diisopropylethylamine (18.0 mg, 0.140 mmol, 4.5 eq) were added. The mixture was allowed to stir at room temperature for 2 h, after which it was concentrated under vacuum, dissolved in 1 mL of methanol, and purified by HPLC using an Alltech Econosil C_{18 10}U 10×250 mm column and acetonitrile 0-95% in water as mobile phase. Compound 1 was obtained as crystalline white solid.

### Synthesis of Derivatives

Furthermore, the chemical procedure used to synthesize Compound 1 can be used to synthesize analogs of this azole tripeptide molecule. Accordingly, the correlated analogs can be prepared by modifying the reagents to include the analog feature. The synthesis can then be performed as described with the correlated analogs.

FIGS. 8A-8E show a generic reaction scheme that can be used to synthesize derivatives of Compound 1. As shown, schemes 1, 2, 3, 4, and 5 are followed, where the azole group can be any of A1, A2, or A3 as described herein. It should be noted that the position of A1 and A3 can be swapped, or the azoles can be in any order. FIG. 8A shows Scheme 1A. FIG. 8B shows Scheme 2A. FIG. 8C shows Scheme 3A. FIG. 8D shows Scheme 4A, FIG. 8E shows Scheme 5A, which results in the azole tripeptide derivative.

An enantiopure (L,L,L) version of the azo tripeptide was obtained. The N-phthalimido-Dha-methyl ester was subjected to nucleophilic addition with 1,2,4-triazole 5 and 5-methyltetrazole, the formation of the azole-containing unnatural amino acids worked well, giving productive amounts of compound. It was also possible to separate the two enantiomers of both N-phthalamido-methyl ester protected peptides by chiral chromatography HPLC, and the L-enantiomer was identified by optical rotation analysis of the free amino acids in both neutral and acidic solution. The L-benzotriazoylalanine was from commercial sources and proceeded to install the relevant protecting groups on the three amino acids and couple them through an LLPS procedure. From this, enantiopure triazole peptide was obtained. This enantiopure triazole peptide (e.g., Compound 1) was used in the CAM assays described herein.

### Therapies

Vasodilators exert their physiological activity by inducing relaxation of vascular smooth muscle, leading to a reduction in vascular resistance and an increase in blood vessel diameter. This dilation of blood vessels results in improved blood flow and reduced systemic or localized blood pressure. The mechanism of action may involve direct interaction with smooth muscle cells or indirect modulation of endogenous signaling pathways, such as increasing nitric oxide availability, enhancing cyclic guanosine monophosphate (cGMP) signaling, or inhibiting calcium influx. By decreasing afterload and preload on the heart, vasodilators improve cardiac output and oxygen delivery to tissues. These agents may target arterial, venous, or both types of vessels, depending on their pharmacological class and molecular structure, thereby providing therapeutic benefit in a variety of cardiovascular, pulmonary, renal, and neurovascular conditions associated with impaired perfusion or elevated vascular tone. The azole tripeptides described herein can be used as vasodilators.

Upon administration of a vasodilator, blood vessels undergo a relaxation of their smooth muscle layer, resulting in dilation or widening of the vessel lumen. This physiological response decreases vascular resistance and allows for increased blood flow through the affected arteries or veins. In arterial vessels, vasodilation reduces systemic blood pressure and lowers the workload on the heart, enhancing perfusion to vital organs and tissues. In venous vessels, dilation increases vascular capacitance, reducing venous return and preload on the heart. The net effect is improved hemodynamic stability and tissue oxygenation, which can alleviate symptoms associated with conditions such as hypertension, ischemia, or heart failure. Depending on the specific agent used, the vasodilation may be selective for arterial, venous, or both vessel types, and may be mediated through molecular pathways involving nitric oxide, prostaglandins, or calcium channel modulation.

Vasodilators primarily cause relaxation of the smooth muscle in the vessel wall, leading to an increase in vessel diameter (vasodilation). The length of the vessel remains essentially unchanged during this process. Vessels are somewhat elastic and can accommodate changes in tone and pressure, but the physiological response to vasodilators is focused on reducing tone and resistance, not altering the vessel's length. Shortening or lengthening of a vessel would involve structural remodeling, which typically occurs over much longer time scales (e.g., in chronic hypertension or during development), not acutely in response to pharmacologic vasodilation.

A method is provided for treating a disorder in a subject in need thereof by administering a therapeutically effective amount of a vasodilator. The disorder to be treated may include, but is not limited to, hypertension, pulmonary arterial hypertension, angina pectoris, congestive heart failure, myocardial ischemia, erectile dysfunction, Raynaud's phenomenon, cerebral vasospasm, peripheral artery disease, coronary artery disease, heart failure with preserved ejection fraction, and vascular dementia. Additional treatable disorders include chronic kidney disease with associated vasoconstriction, preeclampsia, scleroderma-associated vasculopathy, frostbite-associated vasoconstriction, pulmonary fibrosis with vascular involvement, glaucoma, migraine, stroke, claudication, livedoid vasculopathy, systemic sclerosis, altitude sickness, sickle cell disease-associated vasocclusion, and coronary microvascular dysfunction. The vasodilator may be administered alone or in combination with one or more other therapeutic agents, and may be used to treat any one or more of the foregoing disorders. In some aspects, the condition is hypertension, angina, coronary heart disease, heart failure, Raynaud's disease, peripheral artery disease, ischemic bowel disorder, vascular dementia, COVID-19, other ischemic disorders, or combinations thereof. The vasodilator can be an azole tripeptide.

### Biological Data

Stable Angina Pectoris (SAP) is the most prevalent clinical manifestation of Coronary Heart Disease (CHD). The first-line treatment for SAP involves dilating atherosclerotic blood vessels with NO releasing nitrates. However, most nitrates only provide temporary relief from SAP, requiring daily dosing. Repeated use leads to nitrate tolerance and loss of efficacy in as little as 12-24 hours, and the patient must enter a nitrate-free period. During this time, the patient can experience a rebound increase in anginal pain caused by nitrate withdrawal. Precise dosing schedules can offset some of these effects, but only if they are closely adhered to. Long-acting nitrates offer a degree of angina prophylaxis, although an emerging body of evidence now suggests that these agents can cause endothelial dysfunction due to the accumulation of free radicals, increasing the risk of myocardial infarction. Second-line beta-adrenergic blockers can also be used to treat SAP, but these medicines are no more effective than nitrates and carry a greater risk for off target effects at higher concentrations due to poor target specificity. They are generally more useful as antiarrhythmic agents. Third-line calcium channel blockers exhibit high fractions of plasma protein binding and are susceptible to drug-drug interactions. They are also not suitable for patients with liver dysfunction due to their extensive renal metabolism. Thus, it has become increasingly clear that new mechanistically distinct drugs that provide long-lasting vasodilative effects are needed.

In the chorioallantoic membrane assay (CAM), Compound 1 was able to maintain, for 24 hours, an activity comparable to that shown by FDA approved vasodilators after 10 minutes of their administration. This demonstrates its potential to provide long-lasting relief for patients with SAP. Furthermore, its ability to rescue 25% of the chicken embryos exposed to a 100-fold lethal dose of CoCl₂ also displayed its feasibility to lower mortality in CHD patients with chronic total occlusion and heart failure. These findings indicate that Compound 1 and its derivatives can play a role as a lead molecule in the development of new therapeutics that will improve the quality of life for millions of individuals who suffer from chronic stable angina pectoris and related ischemic disorders.

### CAM

Fertilized Brown Leghorn chicken eggs were carefully cleaned with isopropanol and incubated on a Kebonnixs incubator at 37.8° C. and 50-60% relative humidity. At day 2, the egg was carefully cleaned with isopropanol again and a small opening was carved near its air sac through which approximately 3 mL of albumin was removed. The opening was sealed with medical tape and a larger square opening of approximately 1×1 cm was carved near the center of the egg using a Dremer Stylo+ tool. The egg was further cleaned in order to remove debris and the eggshell membrane was carefully removed using sterilized tweezers. The opening was sealed using clear tape and the eggs were incubated for four more days under the same conditions after disabling the auto-turning mechanism of the incubator.

At day 6, 10 μL of a 40 μM solution of Compound 1 (e.g., ARU_IV_21_2) 5 (or controls) was placed on a 6 mm diameter Biogram blank sterile antibiotic sensitivity paper disc. The tape covering the opening in the egg was carefully removed and the disc was carefully placed on top of the chorioallantoic membrane (e.g., CAM) using sterilized tweezers, then the opening was resealed, and the eggs were placed again in the incubator.

After 24 hours, the tape was removed, and the opening was further expanded until all the possible area of the CAM was exposed. The paper disc was carefully removed, and the CAM was photographed. For the Miles assay approximately 100 μl of a 1% w/v solution of Evans Blue dye was injected in the CAM and infiltration of the dye into the blood vessels was analyzed using an Andonstar digital microscope equipped with a 60× magnification lens. If the dye infiltrated the blood vessels the test was considered positive. The photographs were later cropped to include only the CAM and analyzed using the NIH ImageJ software package as follows.

To analyze the extent of branching the blood vessels were carefully traced in order for the ImageJ software to be able to distinguish them for the background. The traced images were opened on the ImageJ software, converted into an 8 bit image and the threshold was adjusted to exclude any possible background signal. This process gave a binary (black and white) map of the vasculature which was then skeletonized (converted into a 1 pixel wide map) and the skeleton was analyzed to determine the number of branches and junction voxels (pixels connected to more than 2 other pixels, i.e. pixels in which the map divides).

To analyze the vascular length density and vascular density the cropped image was opened on the ImageJ software, and the Mexican Hat filter plugin was applied using a radius of 5.0. After this, the image was made binary and the Vascular Density function of the Vessel Analysis plugin was used to determine both parameters after carefully tracing the image so that only the CAM area was included on the calculation.

To analyze the vessel the cropped image was opened on the ImageJ software, and the Mexican Hat filter plugin was applied using a radius of 5.0. After this, the image was made binary, and the Geometry to Distance Map tool of the “Local Distance” tab was applied using a threshold value of 3. The Diameter Measurements function of the Vessel Analysis plugin was used to determine the mean diameter of the blood vessels in the CAM after standardizing the image size to 16.82 pixels=1 mm.

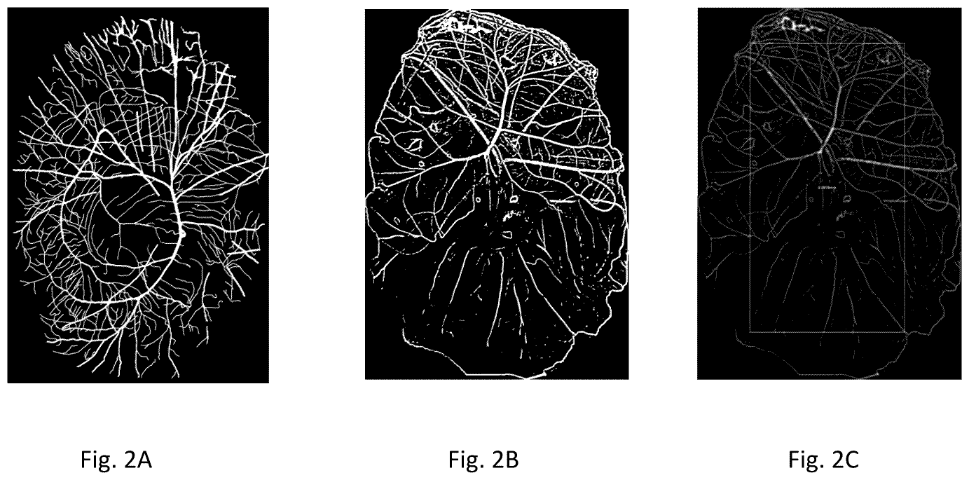

FIG. 2A includes an image that shows a large number of branching points. FIG. 2B includes an image that shows vascularization, with vascular density and length density. FIG. 2C includes an image that shows the vessel thickness, such as average vessel diameter.

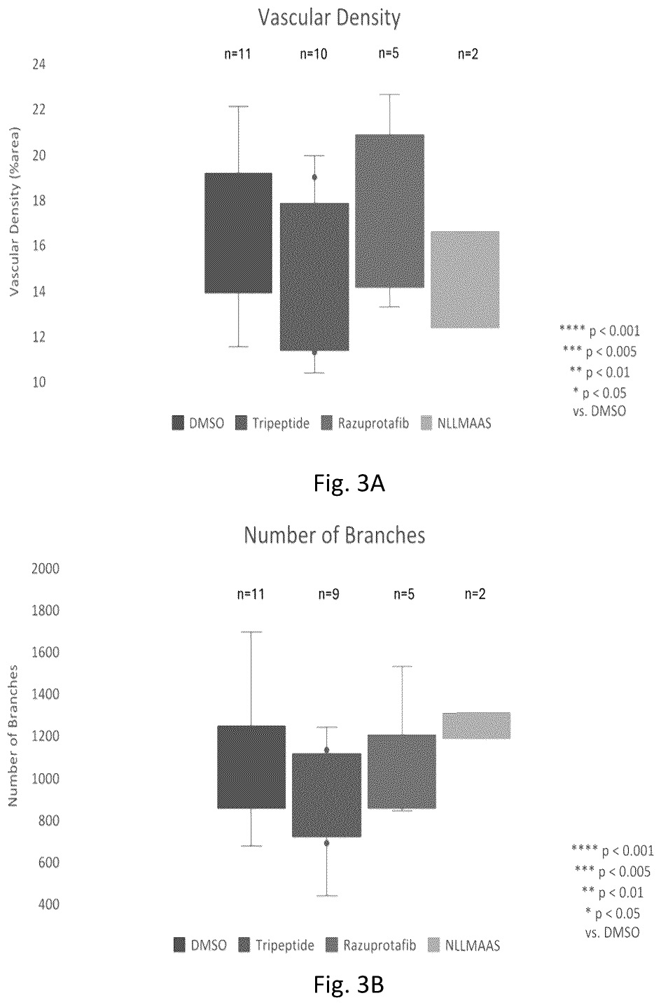

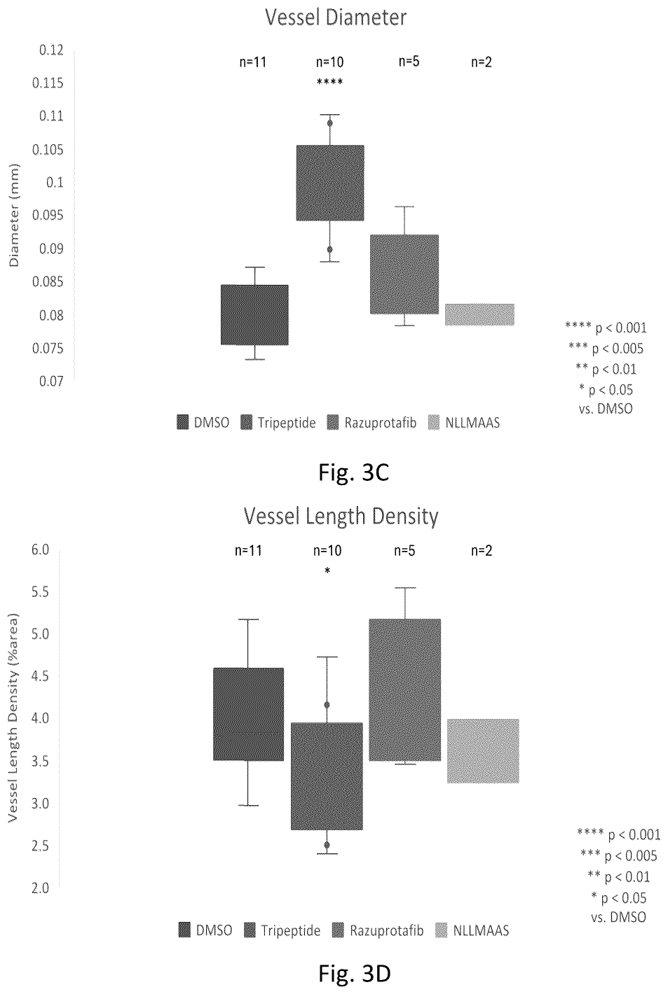

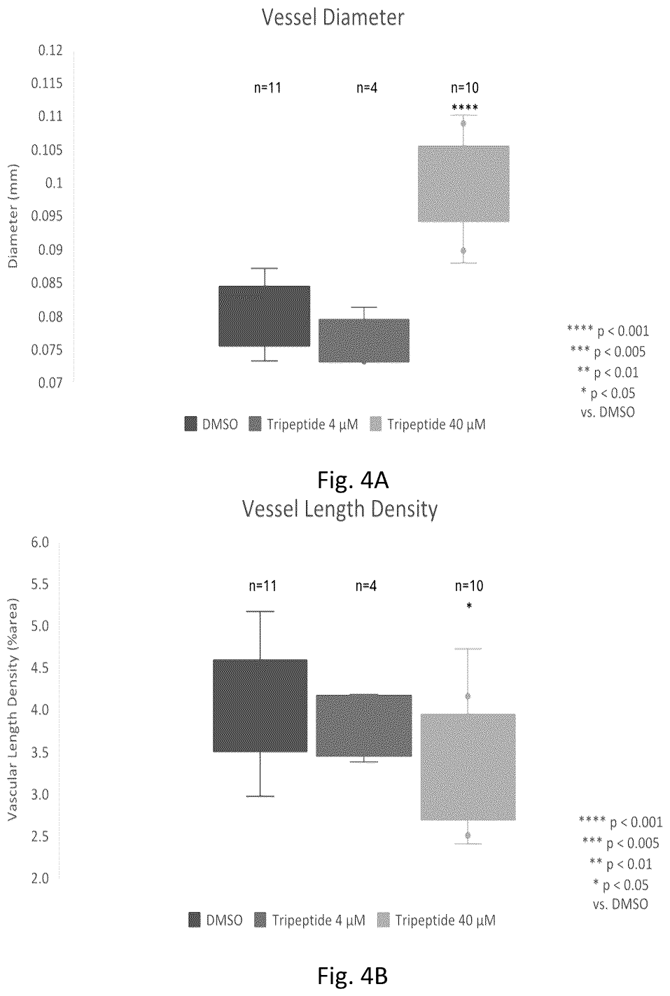

A dose of Compound 2 on day 6 of embryonic development and observe and compare changes in vasculature on day 7 of embryonic development, 24 h after the administration of compounds. The protocol included DMSO as a negative control and Razuprotafib as a positive control. The protocol also included the heptapeptide NLLMAAS as a negative control, which is an orthosteric antagonist of ANG-1. When the protocol administered a 40 mM dose of compounds, which was determined using the original dose at which NLLMAAS was evaluated in the CAM assay, almost all embryos that received Compound 2 or Razuprotafib did not survive 24 h. 40 mM of NLLMAAS (n=2, 1075 mg/Kg) did not have any effects on branching, VD, VLD, or vessel diameter, as compared to DMSO control (n=11). The experiment reduced the dose to 40 μM, and by doing this, no changes were observed in these parameters after the administration of Razuprotafib (n=5, 0.88 mg/Kg) either (again, as compared to DMSO control). The administration of Compound 2 (n=10, 0.91 mg/Kg) did not affect branching or VD; however, it did cause an increase (+124%, p<0.001) in vessel diameter and a slight decrease (17%, p<0.05) in VLD (see, FIGS. 3A, 3B, 3C, and 3D). These effects were lost when a 4 μM dose of KU04212 was administered (n=4, 0.09 mg/Kg) (see, FIGS. 4A and 4B).

FIG. 3A illustrates the vascular density that results from exposure to the Compound 1 tripeptide against controls and angiogenesis modulators, razuprotafib and NLLMAAS heptapeptide. The assay components are in the order of the bars.

FIG. 3B illustrates the number of branches that results from exposure to the Compound 1 tripeptide against controls and angiogenesis modulators, razuprotafib and NLLMAAS heptapeptide. The assay components are in the order of the bars.

FIG. 3C illustrates the vessel diameter that results from exposure to the Compound 1 tripeptide against controls and angiogenesis modulators. The assay components are in the order of the bars.

FIG. 3D illustrates the vessel length density that results from exposure to the Compound 1 tripeptide against controls and angiogenesis modulators, razuprotafib and NLLMAAS heptapeptide. The assay components are in the order of the bars.

FIG. 4A illustrates the vessel diameter that results from exposure to the a 4 μM and 40 μM of Compound 1 tripeptide. The assay components are in the order of the bars.

FIG. 4B illustrates the vessel length density that results from exposure to the a 4 μM and 40 μM of Compound 1 tripeptide. The assay components are in the order of the bars.

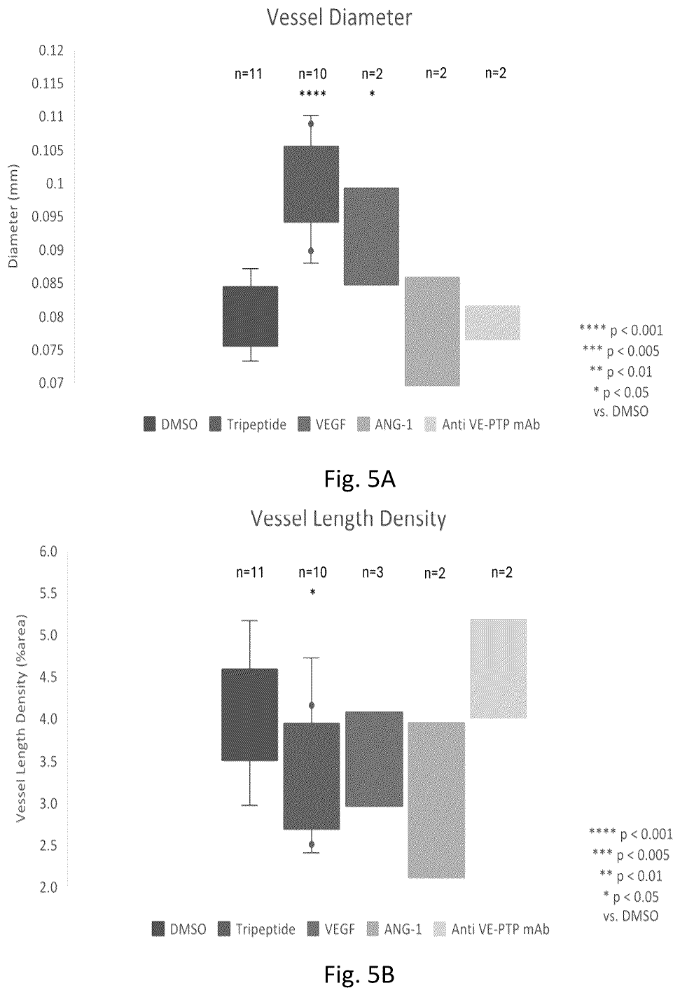

Another study administered three different substances to the CAM of the embryos in order to determine if any of them could recapitulate the action of Compound 2: ANG-1 protein (Gibco, 18.87 μM, n=2, 0.75 mg/Kg), Ms monoclonal antibody to VE-PTP [122.2](Abcam, 1 mg/mL, n=2, 0.75 mg/Kg), and VEGF-165 protein (Gibco, 18.87 μM, n=3, 0.75 mg/Kg). Treatment with any of these compounds did not produce any effects significantly different to those of DMSO controls. This result suggests that Compound 1 may not be acting through the ANG-1/Tie2 pathway and may be instead solely a sustained-action vasodilator (see, FIG. 5A-5B).

FIG. 5A illustrates the vessel diameter that results from exposure to the Compound 1 tripeptide against controls and angiogenesis modulators, VEGF, ANG-1, and ANtiVE-PTP mA B. The assay components are in the order of the bars.

FIG. 5B illustrates the vessel length density that results from exposure to the Compound 1 tripeptide against controls and angiogenesis modulators, VEGF, ANG-1, and ANtiVE-PTP mA B. The assay components are in the order of the bars.

To determine whether Compound 2 exerted its effects through a NO⋅ donation mechanism, a Ca²⁺ channel blockage mechanism, an adrenoreceptor blockage mechanism or an AT1 receptor blockage mechanism, embryos were dosed with isosorbide mononitrate, amlodipine, prazosin and losartan. Recapitulation of the effects of Compound 2 by any of these vasodilators could hint that Compound 1 can have its observed vasodilatory effect by acting though either of these four mechanisms, respectively.

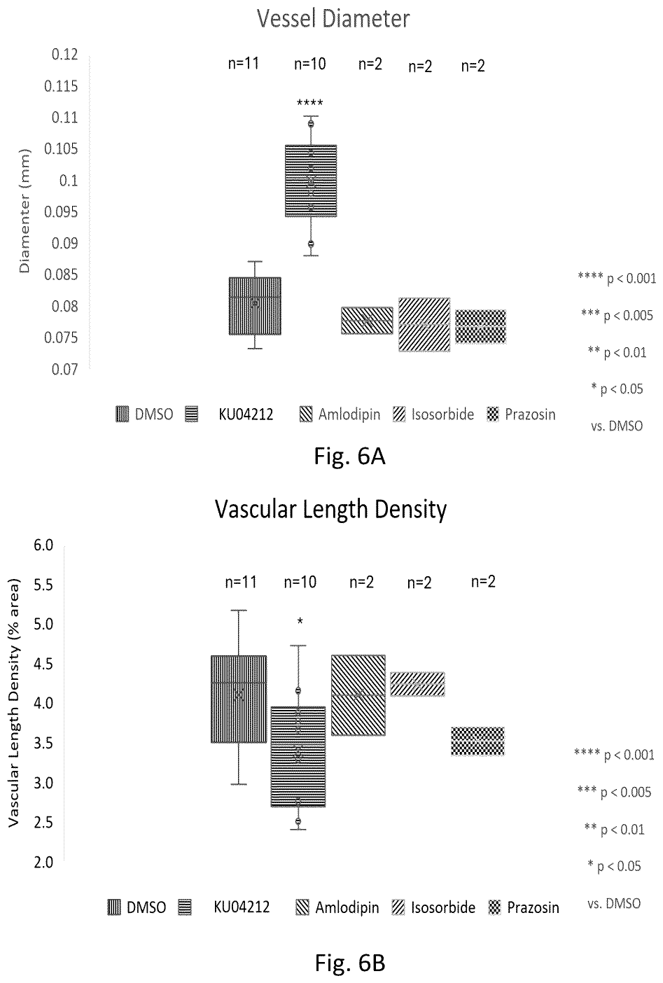

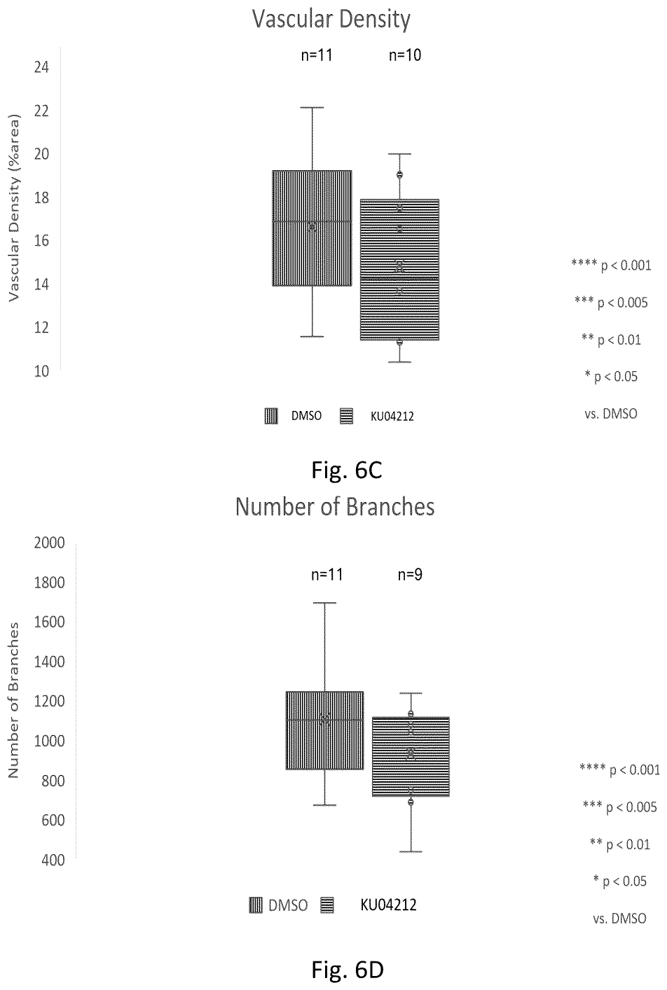

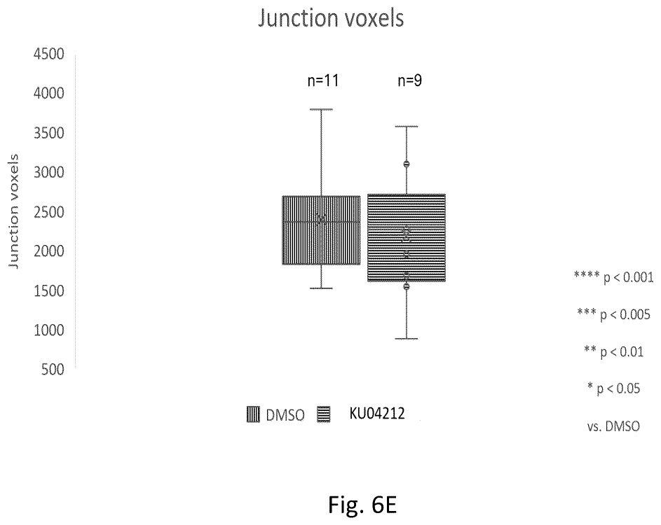

Two out of three of the embryos that received amlodipine (40 μM, n=3, 0.611 mg/Kg) and isosorbide (40 μM, n=3, 0.286 mg/Kg) survived, and all of the embryos that received prazosin (40 μM, n=2, 0.573 mg/Kg) survived. For the case of these three drugs, though, we did not observe effects in VD, VLD, or vessel diameter that were significantly different than DMSO controls after 24 hours. FIGS. 6A-6B illustrate the azo tripeptides, such as Compound 1 (e.g., ARU_IV_21_2), functions as a vasodilator.

FIG. 6A illustrates the vessel diameter that results from exposure to the Compound 1 tripeptide against controls and vasodilators, amlodipine, isosorbide, and prazosin. Vessel diameter in 7-day-old chicken embryos 24 h after administration of 10 μL of a 40 μM solution of Compound 1, model vasodilators, or DMSO control.

FIG. 6B illustrates the vessel length density that results from exposure to the Compound 1 tripeptide against controls and vasodilators, amlodipine, isosorbide, and prazosin. Vessel diameter in 7-day-old chicken embryos 24 h after administration of 10 μL of a 40 μM solution of Compound 1, model vasodilators, or DMSO control.

FIG. 6C illustrates vascular density that results from exposure to the Compound 1 tripeptide against controls and vasodilators, amlodipine, isosorbide, and prazosin. Vessel diameter in 7-day-old chicken embryos 24 h after administration of 10 μL of a 40 μM solution of Compound 1, model vasodilators, or DMSO control.

FIG. 6D illustrates the number of branches for Compound 1 versus the DMSO control. Number of branching points in vessels of 7-day-old chicken embryos 24 h after administration of 10 μL of a 40 μM solution of Compound 1, or DMSO control.

FIG. 6E illustrates junction voxels for Compound 1 versus the DMSO control. Number of junction voxels in skeletonized images of 7-day-old chicken embryos 24 h after administration of 10 μL of a 40 μM solution of Compound 1, or DMSO control.

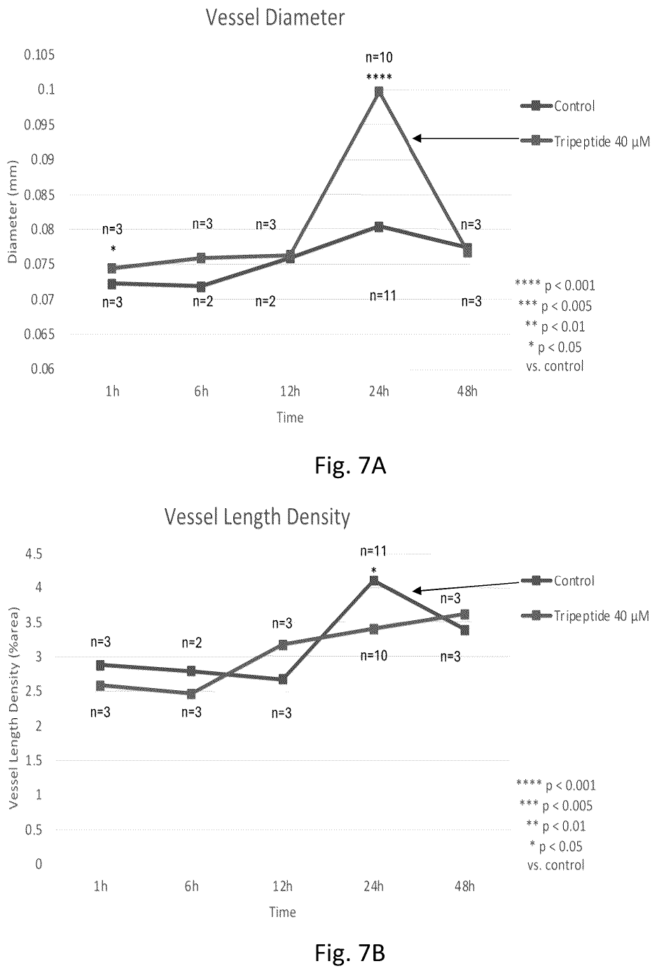

The vessel diameter of vessels in the CAM were measured after 1, 6, 12, 24, and 48 hours of administering Compound 2 (40 μM, 0.91 mg/Kg), and compared it against the diameter of vessels in the CAM after the administration of DMSO, measured at these same time points. From this study, a modest increase in vessel diameter was found at early time points (1 h and 6 h), which was significantly different from to control after 1 h (p<0.05), but not after 6 h; these differences receded at the 12 h time point but then the greatest increase in vessel diameter occurred after 24 h of compound administration, and finally the vessel diameter receded back to baseline levels after 48 h of compound administration (See, FIGS. 7A-7B). The Compound 2 has some modest vasodilatory activity that can be observed after 1 h (and, to some extent, even 6 h) of compound administration, but the major vasodilatory effect was observed after 24 hours of compound administration comes from a metabolic product of Compound 2.

FIG. 7A shows a graph of the vessel diameter that results for control (flat) versus the tripeptide at 40 μM (having peak) over time.

FIG. 7B shows a graph of the vessel length density that results for control (flat) versus the tripeptide at 40 μM (having peak) overtime.

Additionally, an assessment of vascular permeability of the Compound 1 tetrapeptide, which shows sufficient vascular permeability but did not increase permeability. Pharmacokinetic assays show that Compound 1 has plasma stability that results in 87% conversion of the terminal methoxy to a terminal hydroxyl after 2 hours. Microsomal stability was determined to be 48% conversion of the terminal methoxy to a terminal hydroxyl after 24 hours. There was also 34% conversion to another structure having an aldehyde for A² after 24 hours.

### Definitions

By “substituted” as in “substituted alkyl,” “substituted aryl,” and the like, as alluded to in some of the definitions provided herein, is meant that in the alkyl, aryl, or other moiety, at least one hydrogen atom bound to a carbon (or other) atom is replaced with one or more non-hydrogen substituents.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

As used herein, “optionally substituted” indicates that a chemical structure may be optionally substituted with a substituent group, such as defined herein. That is, when a chemical structure includes an atom that is optionally substituted, the atom may or may not include the optional substituent group, and thereby the chemical structure may be considered to be substituted when having a substituent on the atom or unsubstituted when omitting a substituent from the atom. A substituted group, referred to as a “substituent” or “substituent group”, can be coupled (e.g., covalently) to a previously unsubstituted parent structure, wherein one or more hydrogens atoms (or other substituent groups) on the parent structure have been independently replaced by one or more of the substituents. The substituent is a chemical moiety that is added to a base chemical structure, such as a chemical scaffold. As such, a substituted chemical structure may have one or more substituent groups on the parent structure, such as by each substituent group being coupled to an atom of the parent structure. The substituent groups that can be coupled to the parent structure can be any possible substituent group. In examples of the present technology, the substituent groups (e.g., R groups) can be independently selected from an alkyl, —O-alkyl (e.g. —OCH₃, —OC₂H₅, —OC₃H₇, —OC₄H₉, etc.), —S-alkyl (e.g., —SCH₃, —SC₂H₅, —SC₃H₇, —SC₄H₉, etc.), —NR′R″, —OH, —SH, —CN, —NO₂, or a halogen, wherein R′ and R″ are independently H or an optionally substituted alkyl. Wherever a substituent is described as “optionally substituted,” that substituent can also be optionally substituted with the above substituents.

The term “alkyl” or “aliphatic” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, or 1 to about 12 carbon atoms. The term “lower alkyl” intends an alkyl group of 1 to 6 carbon atoms. Substituents identified as “C₁-C₆ alkyl” or “lower alkyl” contains 1 to 3 carbon atoms, and such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The terms “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, or 2 to 12 carbon atoms. The term “lower alkenyl” intends an alkenyl group of 2 to 6 carbon atoms, and the specific term “cycloalkenyl” intends a cyclic alkenyl group, or having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, or 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein contain 1 to 3 carbon atoms, and such substituents contain 1 or 2 carbon atoms (i.e., methoxy and ethoxy).

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Examples of aryl groups contain 5 to 20 carbon atoms, and aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Examples of aryloxy groups contain 5 to 20 carbon atoms, and aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” areas defined above. Examples of aralkyl groups contain 6 to 24 carbon atoms, and aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethyinaphthyl, 7-cyclooctylnaphthyl, 3-ethyl-cyclopenta-1,4-diene, and the like.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, and fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “hydrocarbyl” refers to univalent hydrocarbyl radicals containing 1 to about 30 carbon atoms, or 1 to about 24 carbon atoms, or 1 to about 18 carbon atoms, or about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

All other chemistry terms are defined as known in the art.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

### REFERENCES

The Day 6 embryo has a weight of 267.5+9.7 mg. See: Hu, N.; Clark, E. B. Hemodynamics of the stage 12 to stage 29 chick embryo. Circ. Res. 1989, 65, 1665-1670.
Swaminathan, A.; Balaguru, U. M.; Manjunathan, R.; Bhuvaneswari, S.; Kasiviswanathan, D.; Sirishakalyani, B.; Nayak, P.; Chatterjee, S. Live imaging and analysis of vasoactive properties of drugs using an in-ovo chicken embryo model: replacing and reducing animal testing. Microscopy and Microanalysis 2019, 25, 961-970.
Tay, S. L. M.; Heng, P. W. S.; Chan, L. W. The chick chlorioallantoic membrane imaging method as a platform to evaluate vasoactivity and assess irritancy of compounds. i. Pharm. Pharmacol. 2012, 64, 1128-1137.
Suryapranata, H.; Serruys, P. W.; de Feyter, P. J. Coronary vasodilatory action after a single dose of nicorandil. Am. i. Cardiol. 1988, 61, 292-297.
Tournaire, R.; Simon, M.-P.; le Noble, F.; Eichmann, A.; England, P.; Pouyssegur, J. A. short synthetic peptide inhibits signal transduction, migration and angiogenesis mediated by Tie2 receptor. EMBO Rep. 2004, 5, 262-267.
Lin, S.; Lin, R.; Zhang, H.; Xu, Q.; He, Y. Peripheral vascular remodeling during ischemia. Front. Pharmacol. 2022, 13, 1078047
Bagi, Z.; Brandner, D. D.; Le, P.; McNeal, D. W.; Gong, X. et al. Vasodilator dysfunction and oligodendrocyte dysmaturation in aging white matter. Ann. Neurol. 2018, 83, 142-152.
Pawar, S. S.; Wilcox, E. M.; van Haren, F. M. P. Inhaled pulmonary vasodilators in severe COVID-19: don't hold your breath. i. Crit. Care 2022, 69, 153988.
Daiber, A.; Munzel, T. Organic nitrate therapy, nitrate tolerance, and nitrate-induced endothelial dysfunction: emphasis on redox biology and oxidative stress. Antioxid. Redox Signal 2015, 23, 899-942.
Boden, W. E.; Finn, A. V.; Patel, D.; Peacock, W. F.; Thadani, U.; Zimmerman, F. H. Nitrates as an integral part of optimal medical therapy and cardiac rehabilitation for stable angina: review of current concepts and therapeutics. Clin. Cardiol. 2012, 35, 263-271.
Tarkin, J. M.; Kaski, J. C. Vasodilator therapy: nitrates and nicorandil. Cardiovasc. Drugs Ther. 2016, 30, 367-378.
Kim, H.-J.; Jo, S.-H.; Lee, M.-H.; Seo, W.-W.; Kim, H.-L. et al. Nitrates vs. other types of vasodilators and clinical outcomes in patients with vasospastic angina: a propensity score-matched analysis. i. Clin. Med. 2022, 11, 3250.
Shu, D. F.; Dong, B. R.; Lin, X. F.; Wu, T. X.; Liu, G. J. Long-term beta blockers 5 for stable angina: systematic review and meta-analysis. Eur. i. Preven. Cardiol. 2012, 19, 330-341.
Heupler, F. A. Calcium blockers in angina. How they work, when to prescribe. Postgrad. Med. 1984, 75, 127-32.
Yusuf, S. Calcium antagonists in coronary artery disease and hypertension. Time for reevaluation? Circulation 1995, 92, 1079-1082.
Sueta, D.; Tabata, N.; Hokimoto, S. Clinical roles of calcium channel blockers in ischemic heart diseases. Hypertension Research 2017, 40, 423-428.

1. A compound comprising a structure of Formula 1, Formula 1A, or Formula 1B, or derivative thereof, prodrug thereof, salt thereof, stereoisomer thereof, or having any chirality at any chiral center, or tautomer, polymorph, solvate, or combination thereof, 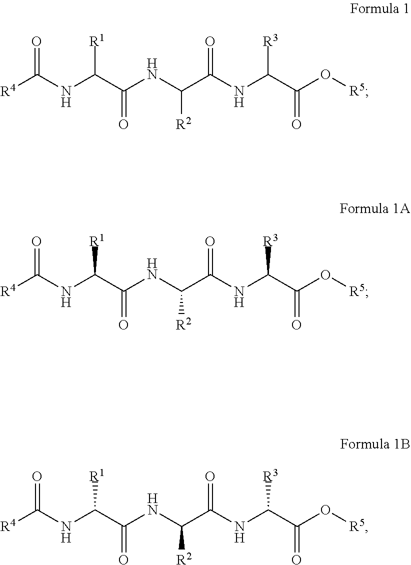
wherein:
R¹, R², and R³ each independently include a substituent having at least one azole group that is substituted or unsubstituted; and
R⁴ and R⁵ each independently include end cap moieties.

2. The compound of claim 1, comprising a structure of Formula 2, Formula 2A, or Formula 2B, or derivative thereof, prodrug thereof, salt thereof, stereoisomer thereof, or having any chirality at any chiral center, or tautomer, polymorph, solvate, or combination thereof, 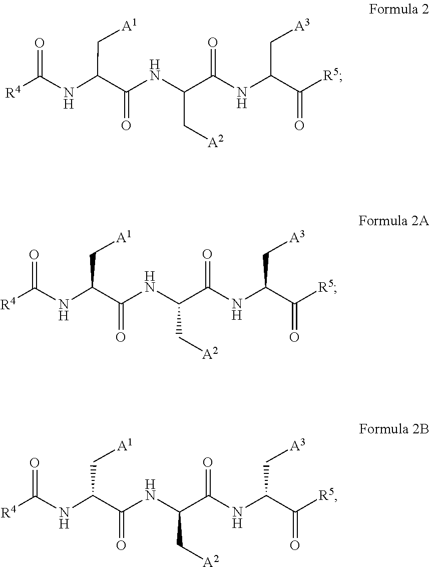
wherein:
A¹, A², and A³ are each independently azole group that is substituted or unsubstituted.

3. The compound of claim 2, wherein each azole group is independently a monocyclic or fused bicyclic heteroaryl groups having 5 to 10 ring atoms containing one to four heteroatoms independently selected from nitrogen, oxygen, and sulfur.

4. The compound of claim 3, wherein each azole group is independently selected from pyrrole, imidazole, pyrazole, triazole (1,2,3- and 1,2,4-isomers), tetrazole, oxazole, isoxazole, thiazole, isothiazole, and benzofused analogs such as benzoxazole, benzimidazole, benzothiazole, indole, indazole, carbazole, isoindole, benzotriazole, and combinations thereof.

5. The compound of claim 4, wherein R⁴ includes methyl, ethyl, n-propyl, isopropyl, n-butyl, sec-butyl, tert-butyl, pentyl, hexyl, cyclopropyl, cyclobutyl, cyclopentyl, cyclohexyl, phenyl, benzyl, tolyl, xylyl, anisyl, naphthyl, methoxy, ethoxy, propoxy, isopropoxy, tert-butoxy, phenoxy, benzoxy, 4-methoxyphenyl, 4-nitrophenyl, acetyl, propionyl, butyryl, isobutyryl, pivaloyl, benzoyl, trifluoroacetyl, methanesulfonyl, trifluoromethanesulfonyl, benzenesulfonyl, tosyl, brosyl, nosyl, trimethylsilyl, triethylsilyl, tert-butyldimethylsilyl, triisopropylsilyl, imidazolyl, triazolyl, tetrazolyl, morpholinyl, piperidinyl, piperazinyl, pyridyl, dimethylamino, diethylamino, morpholino, methyl carbonate, ethyl carbonate, phenyl carbonate, methyl carbamate, ethyl carbamate, benzyl carbamate, tert-butyl carbamate, and combinations thereof.

6. The compound of claim 5, wherein R⁵ includes methyl ester, ethyl ester, n-propyl ester, isopropyl ester, tert-butyl ester, pentyl ester, benzyl ester, phenyl ester, methylamide, ethylamide, isopropylamide, tert-butylamide, benzylamide, anilide, morpholine amide, piperidine amide, pyrrolidine amide, alkyl-substituted urea, aryl-substituted urea, alkyl-substituted thiourea, aryl-substituted thiourea, methanesulfonamide, trifluoromethanesulfonamide, benzenesulfonamide, trimethylsilyl ester, tert-butyldimethylsilyl amide, and combinations thereof.

7. The compound of claim 1, comprising a structure of Formula 3, Formula 3A, or Formula 3B, or derivative thereof, prodrug thereof, salt thereof, stereoisomer thereof, or having any chirality at any chiral center, or tautomer, polymorph, solvate, or combination thereof, 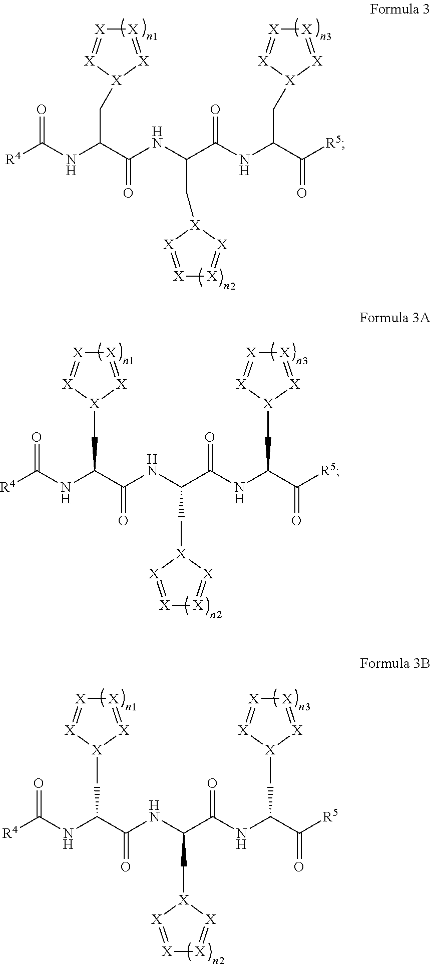
wherein:
each X is independently carbon, nitrogen, oxygen, and sulfur, so long as one X is carbon and one X is nitrogen, and wherein each X that is carbon is substituted or unsubstituted, wherein adjacent carbons of each azole group can include a cycle fused with the respective azole group;
n1, n2, and n3 are each independently an integer; and
each linking X is a carbon or a nitrogen.

8. The compound of claim 1, comprising a structure of Formula 4, Formula 4A, or Formula 4B, or derivative thereof, prodrug thereof, salt thereof, stereoisomer thereof, or having any chirality at any chiral center, or tautomer, polymorph, solvate, or combination thereof, 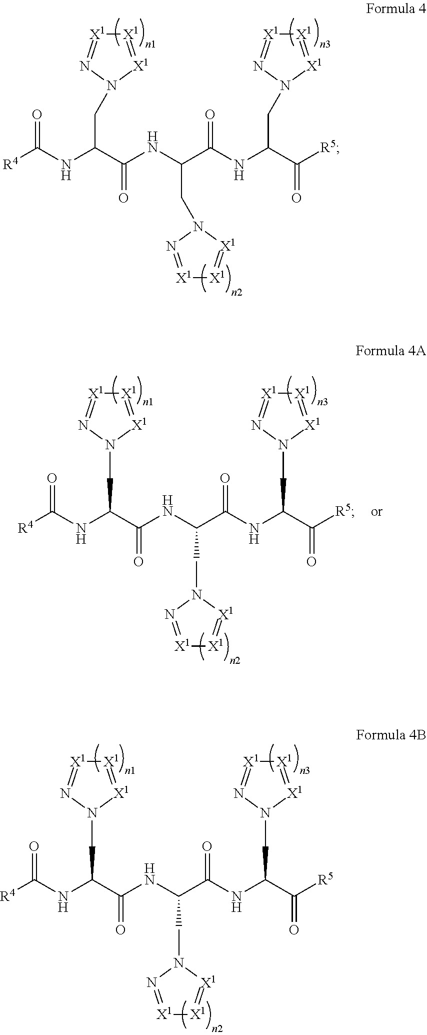
wherein:
each X¹ is independently carbon or nitrogen, so long as at least one X¹ is carbon, and wherein each X¹ that is carbon is substituted or unsubstituted, wherein adjacent carbons of each azole group can include a cycle fused with the respective azole group; and
n1, n2, and n3 are each independently an integer.

9. The compound of claim 1, comprising a structure of Formula 5, Formula 5A, or Formula 5B, or derivative thereof, prodrug thereof, salt thereof, stereoisomer thereof, or having any chirality at any chiral center, or tautomer, polymorph, solvate, or combination thereof, 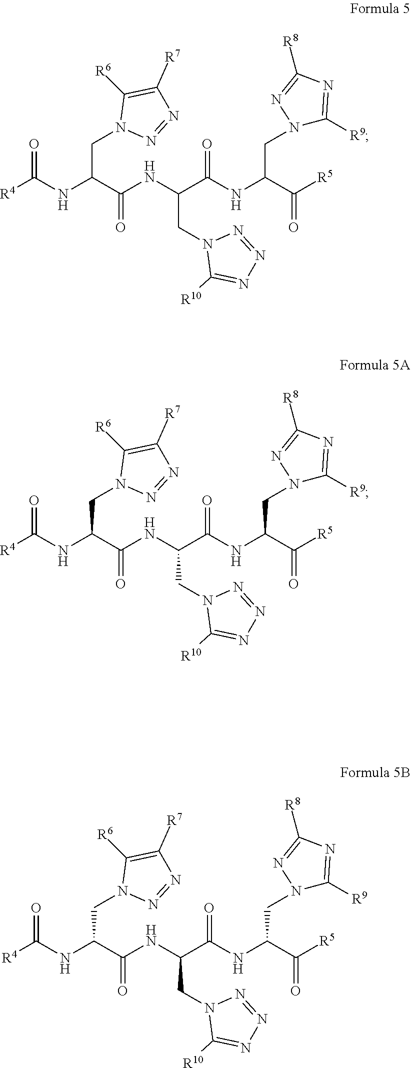
wherein:
R⁶ and R⁷ are independently a hydrogen or a substituent, wherein adjacent carbons of each azole group can include R⁶ and R⁷ forming a cycle fused with the respective azole group;
R⁸, R⁹, and R¹⁰ can independently be hydrogen or a substituent; and
n1, n2, and n3 are each independently an integer.

10. The compound of claim 9, wherein R⁴ includes methyl, ethyl, n-propyl, isopropyl, n-butyl, sec-butyl, tert-butyl, pentyl, hexyl, cyclopropyl, cyclobutyl, cyclopentyl, cyclohexyl, phenyl, benzyl, tolyl, xylyl, anisyl, naphthyl, methoxy, ethoxy, propoxy, isopropoxy, tert-butoxy, phenoxy, benzoxy, 4-methoxyphenyl, 4-nitrophenyl, acetyl, propionyl, butyryl, isobutyryl, pivaloyl, benzoyl, trifluoroacetyl, methanesulfonyl, trifluoromethanesulfonyl, benzenesulfonyl, tosyl, brosyl, nosyl, trimethylsilyl, triethylsilyl, tert-butyldimethylsilyl, triisopropylsilyl, imidazolyl, triazolyl, tetrazolyl, morpholinyl, piperidinyl, piperazinyl, pyridyl, dimethylamino, diethylamino, morpholino, methyl carbonate, ethyl carbonate, phenyl carbonate, methyl carbamate, ethyl carbamate, benzyl carbamate, tert-butyl carbamate, and combinations thereof; and
R⁵ includes methyl ester, ethyl ester, n-propyl ester, isopropyl ester, tert-butyl ester, pentyl ester, benzyl ester, phenyl ester, methylamide, ethylamide, isopropylamide, tert-butylamide, benzylamide, anilide, morpholine amide, piperidine amide, pyrrolidine amide, alkyl-substituted urea, aryl-substituted urea, alkyl-substituted thiourea, aryl-substituted thiourea, methanesulfonamide, trifluoromethanesulfonamide, benzenesulfonamide, trimethylsilyl ester, tert-butyldimethylsilyl amide, and combinations thereof.

11. The compound of claim 10, wherein R⁶, R⁷, R⁸, R⁹, and R¹⁰ are each independently a hydrogen or a substituent, wherein the substituent includes alkyl, alkenyl, alkynyl, cycloalkyl, cycloalkenyl, cycloalkynyl, aryl, polyaryl, hetroaryl, polyhetroaryl, alkaryl, aralkyl, halo, halo-substituted alkyl, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof, and/or
R⁶ and R⁷ form a cycle fused with the respective azole group, wherein the cycle is aliphatic or aromatic.

12. The compound of claim 10, wherein R⁶, R⁷, R⁸, R⁹, and R¹⁰ are each independently a hydrogen or a substituent, wherein the substituent includes C₁-C₂₄alkyl, C₂-C₂₄alkenyl, C₂-C₂₄alkynyl, C₃-C₂₄cycloalkyl, C₄-C₂₄ cycloalkenyl, C₅-C₂₄ cycloalkynyl, C₅-C₂₀ aryl, C₆-C₂₄alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl, acyloxy, C₂-C₂₄ alkoxycarbonyl, C₆-C₂₀ aryloxycarbonyl, halocarbonyl, C₂-C₂₄ alkylcarbonato, C₆-C₂₀ arylcarbonato, carboxy, carboxylato, carbamoyl, mono-(C₁-C₂₄alkyl)-substituted carbamoyl, di-(C₁-C₂₄alkyl)-substituted carbamoyl, mono-substituted arylcarbamoyl, di-substituted arylcarbamoyl, thiocarbamoyl, mono-(C₁-C₂₄alkyl)-substituted thiocarbamoyl, di-(C₁-C₂₄alkyl)-substituted thiocarbamoyl, mono-substituted arylthiocarbamoyl, di-substituted arylthiocarbamoyl, carbamido, mono-(C₁-C₂₄alkyl)-substituted carbamido, di-(C₁-C₂₄alkyl)-substituted carbamido, mono-substituted aryl carbamido, di-substituted aryl carbamido, carbamate, alkyl carbamate, cyano, isocyano, cyanato, isocyanato, thiocyanato, isothiocyanato, azido, formyl, thioformyl, amino, mono- and di-(C₁-C₂₄alkyl)-substituted amino, mono- and di-(C₆-C₂₀ aryl)-substituted amino, C₂-C₂₄alkylamido, C₅-C₂₀ arylamido, imino, alkylimino, arylimino, nitro, nitroso, sulfonic acid, sulfonato, C₁-C₂₄alkylsulfanyl, C₅-C₂₀ arylsulfanyl, C₁-C₂₄alkylsulfinyl, C₅-C₂₀ arylsulfinyl, C₁-C₂₄alkylsulfonyl, C₅-C₂₀ arylsulfonyl, phosphono, phosphonato, phosphinato, phospho, phosphino, phosphate, sulphate, any aryl or cyclo with or without hetero atoms, each being substituted or unsubstituted, and combinations thereof, and/or
R⁶ and R⁷ form a cycle fused with the respective azole group, wherein the cycle is aliphatic or aromatic.

13. The compound of claim 12, wherein the cycle is:
an aromatic group selected from phenyl, naphthyl, anthracenyl, phenanthryl, biphenyl, terphenyl, and fluorenyl; or
an aliphatic group selected from cyclopropyl, cyclobutyl, cyclopentyl, cyclohexyl, cycloheptyl, cyclooctyl, cyclononyl, cyclodecyl, cycloundecyl, cyclododecyl, bicyclo[1.1.0]butyl, bicyclo[2.2.1]heptyl (norbornyl), bicyclo[2.2.2]octyl, adamantyl, and noradamantyl.

14. The compound of claim 1, comprising the structure of:

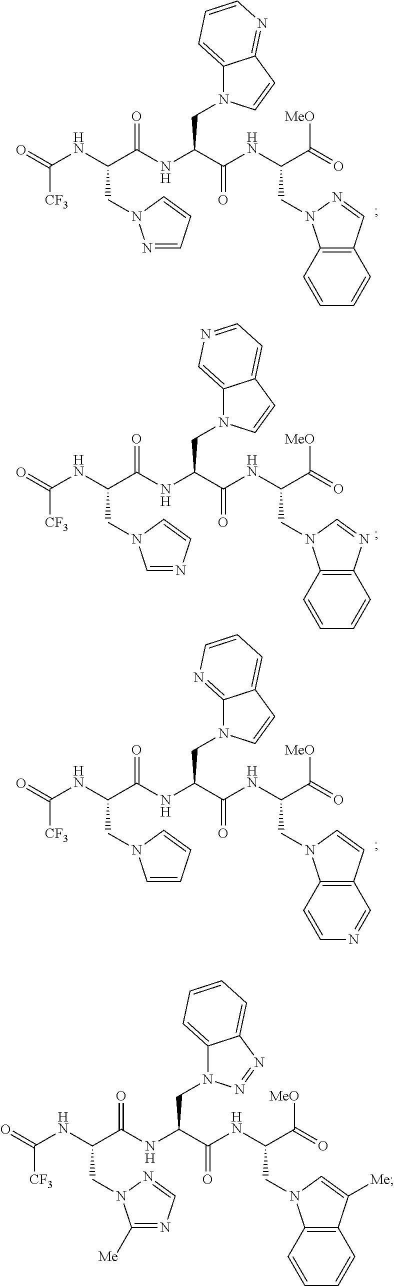 [SMILES: COC(=O)[C@H](Cn1ncc2ccccc21)NC(=O)[C@H](Cn1ccc2ncccc21)NC(=O)[C@H](Cn1cccn1)NC(=O)C(F)(F)F ; COC(=O)[C@H](Cn1cnc2ccccc21)NC(=O)[C@H](Cn1ccc2ccncc21)NC(=O)[C@H](Cn1ccnc1)NC(=O)C(F)(F)F ; COC(=O)[C@H](Cn1ccc2cnccc21)NC(=O)[C@H](Cn1ccc2cccnc21)NC(=O)[C@H](Cn1cccc1)NC(=O)C(F)(F)F ; COC(=O)[C@H](Cn1cc(C)c2ccccc21)NC(=O)[C@H](Cn1nnc2ccccc21)NC(=O)[C@H](Cn1ncnc1C)NC(=O)C(F)(F)F]

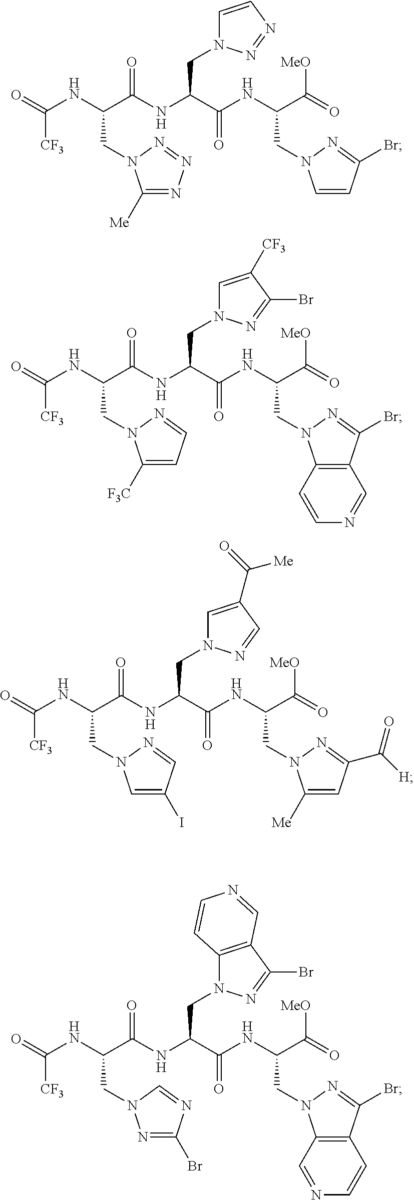

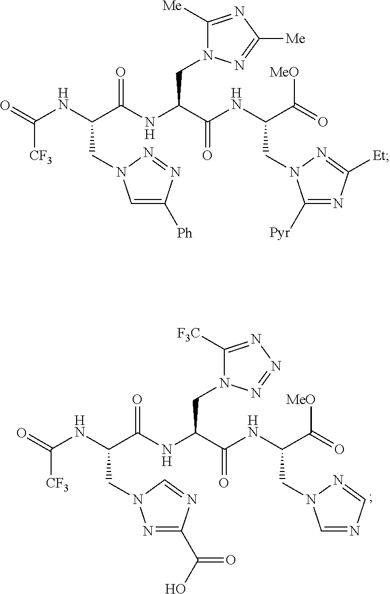

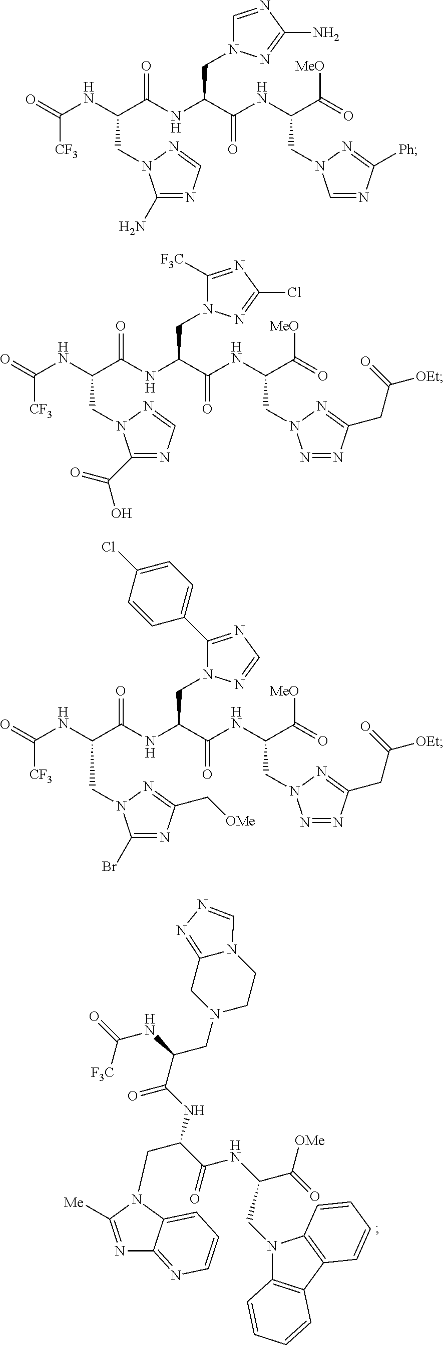

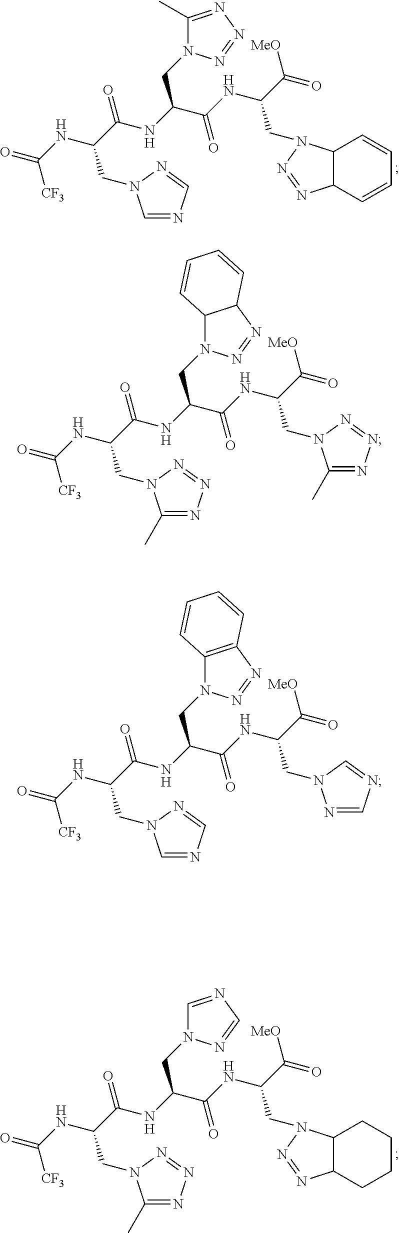

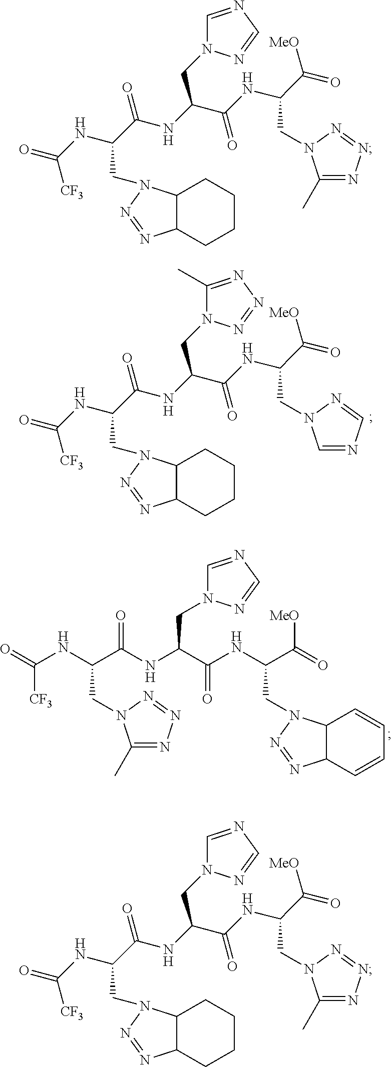

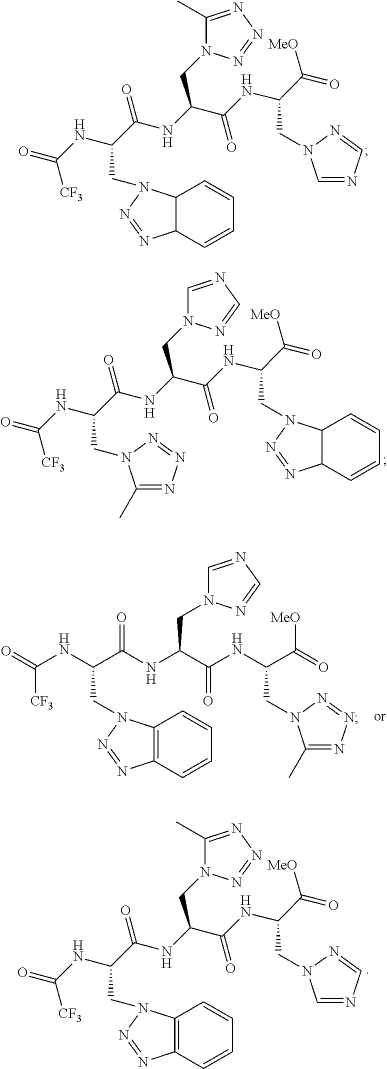

15. The compound of claim 1, wherein the compound consists of an azole tripeptide.

16. A composition comprising the compound of claim 1 and a carrier.

17. A pharmaceutical composition composing the compound of claim 1 and a pharmaceutically acceptable carrier.

18. A method of vasodilation, comprising:
providing a subject; and
administering the compound of claim 1 to the subject in a therapeutically effective amount to cause vasodilation in the subject.

19. A method of treatment of a condition in a subject, comprising:
administering the compound of claim 1 to the subject in a therapeutically effective amount to cause vasodilation in the subject to provide the treatment of the condition.

20. The method of claim 19, wherein the condition is selected from hypertension, pulmonary arterial hypertension, angina pectoris, congestive heart failure, myocardial ischemia, erectile dysfunction, Raynaud's phenomenon, cerebral vasospasm, peripheral artery disease, coronary artery disease, heart failure with preserved ejection fraction, and vascular dementia, chronic kidney disease with associated vasoconstriction, preeclampsia, scleroderma-associated vasculopathy, frostbite-associated vasoconstriction, pulmonary fibrosis with vascular involvement, glaucoma, migraine, stroke, claudication, livedoid vasculopathy, systemic sclerosis, altitude sickness, sickle cell disease-associated vasocclusion, and coronary microvascular dysfunction.

21. The method of claim 19, wherein the subject has a condition that is treated with the compound, wherein the condition is hypertension, angina, coronary heart disease, heart failure, Raynaud's disease, peripheral artery disease, ischemic bowel disorder, vascular dementia, COVID-19, other ischemic disorders, or combinations thereof.

22. A method of preparing an azole tripeptide, comprising:
synthesizing the compound of claim 1.

23. The method of claim 22, further comprising:
coupling an azole molecule with an oxoisoindolinyl acrylate to obtain an azole oxoisoindolinyl ester;
reacting the azole oxoisoindolinyl ester with acid to remove the oxoisoindolinyl and obtain a first azole peptide with a first azole group;
reacting the first azole peptide with methyl alcohol to obtain an azole peptide methyl ester;
obtaining a second azole peptide with a second azole group, wherein the second azole group is the same or different from the first azole group;
reacting the second azole peptide with a nitrogen protecting group to obtain an nitrogen protected azole peptide;
obtaining a third azole peptide with a third azole group, wherein the third azole group is the same or different from the first azole group or the second azole group;
reacting the third azole peptide with a trihaloacetic anhydride to obtain an azole trifluoromethylamide;
reacting the azole peptide methyl ester with the nitrogen protected azole peptide to form nitrogen protected azole dipeptide;
deprotecting the nitrogen protected azole dipeptide to obtain dipeptide with methyl ester; and
reacting dipeptide with methyl ester with azole trifluoromethylamide to obtain the Compound 1.

24. The method of claim 23, further comprising:
performing scheme 1; 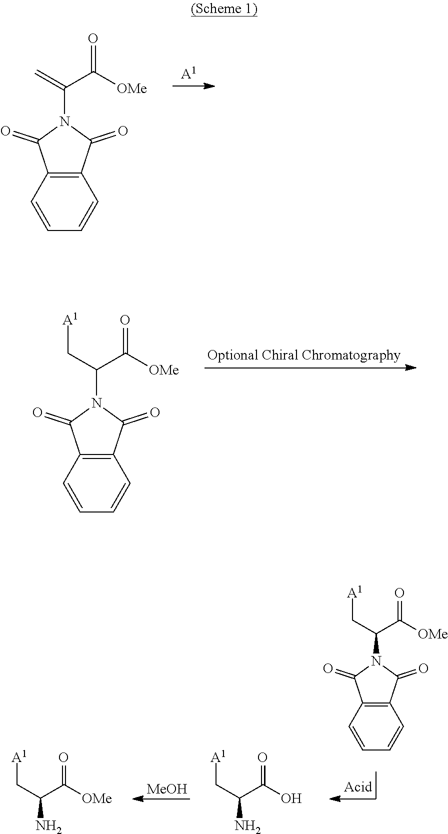
performing scheme 2; 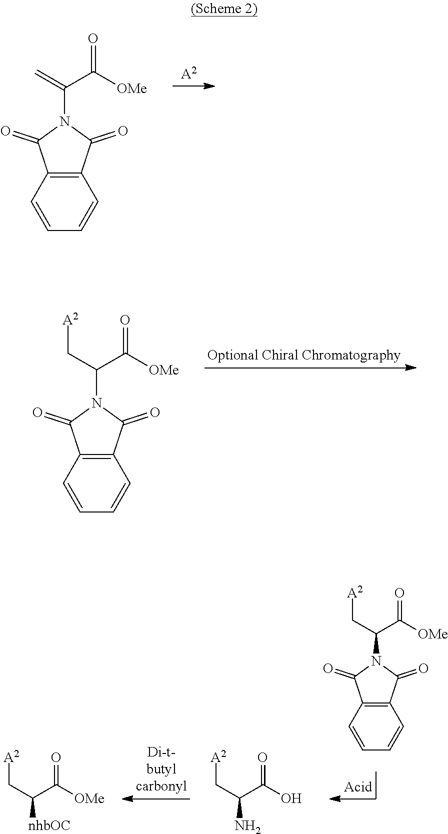
performing scheme 3; 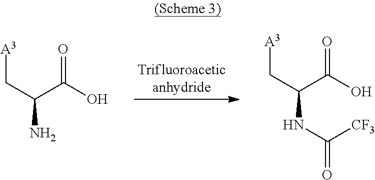
performing scheme 4; 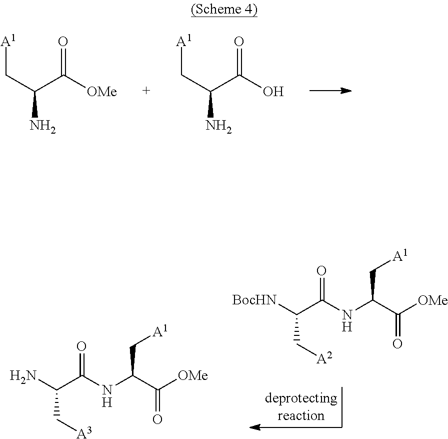
and performing scheme 5;

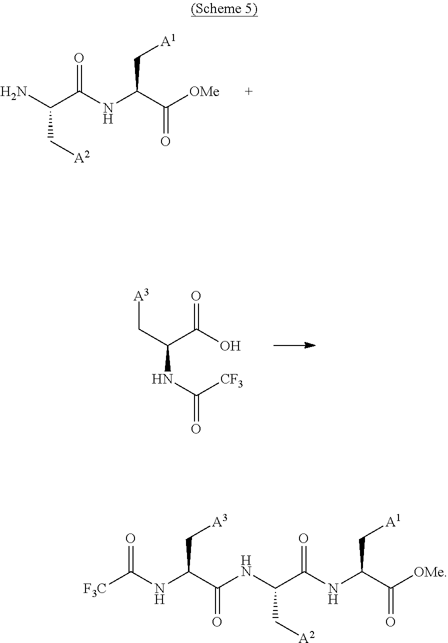

25. The method of claim 24, further comprising:
performing scheme 1; 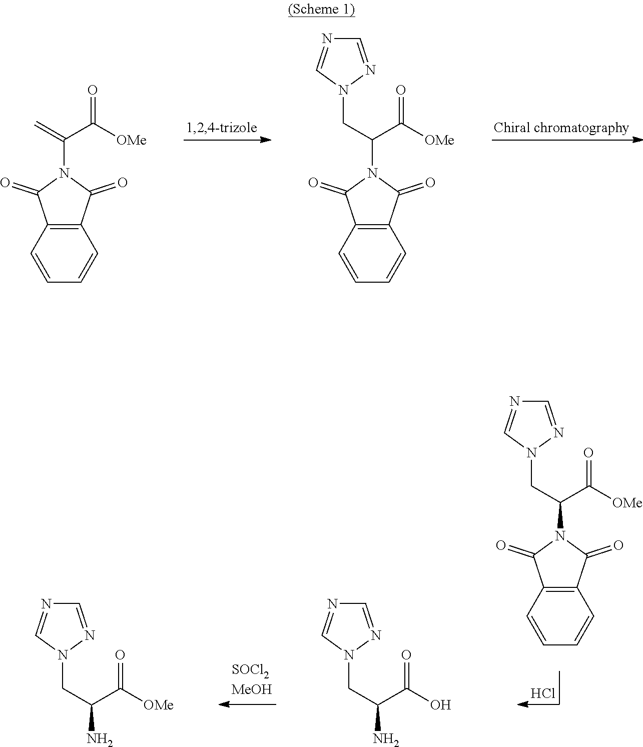 [SMILES: C=C(C(=O)OC)N1C(=O)c2ccccc2C1=O ; COC(=O)C(Cn1cncn1)N1C(=O)c2ccccc2C1=O ; COC(=O)[C@@H](N)Cn1cncn1 ; N[C@@H](Cn1cncn1)C(=O)O ; COC(=O)[C@H](Cn1cncn1)N1C(=O)c2ccccc2C1=O ; Cl]
performing scheme 2; 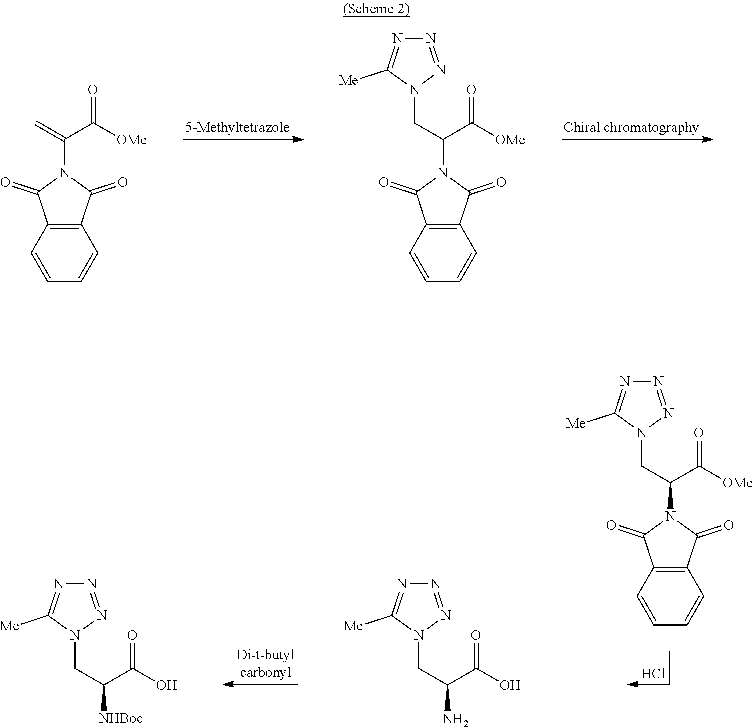 [SMILES: C=C(C(=O)OC)N1C(=O)c2ccccc2C1=O ; COC(=O)C(Cn1nnnc1C)N1C(=O)c2ccccc2C1=O ; Cc1nnnn1C[C@H](NC(=O)OC(C)(C)C)C(=O)O ; Cc1nnnn1C[C@H](N)C(=O)O ; COC(=O)[C@H](Cn1nnnc1C)N1C(=O)c2ccccc2C1=O ; Cl]
performing scheme 3; 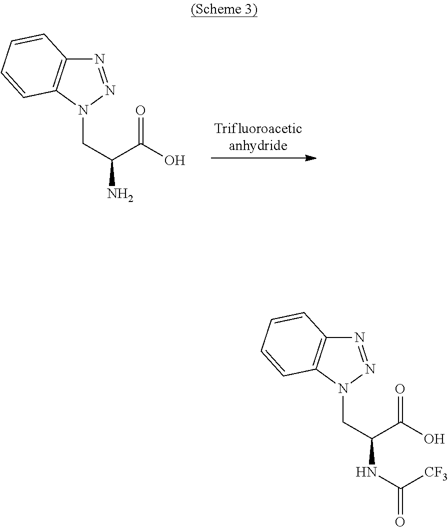 [SMILES: N[C@@H](Cn1nnc2ccccc21)C(=O)O ; O=C(O)[C@H](Cn1nnc2ccccc21)NC(=O)C(F)(F)F]
performing scheme 4; 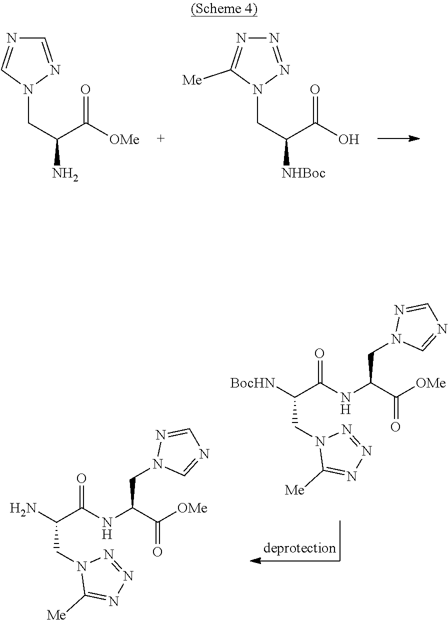 [SMILES: COC(=O)[C@@H](N)Cn1cncn1 ; Cc1nnnn1C[C@H](NC(=O)OC(C)(C)C)C(=O)O ; COC(=O)[C@H](Cn1cncn1)NC(=O)[C@@H](N)Cn1nnnc1C ; COC(=O)[C@H](Cn1cncn1)NC(=O)[C@H](Cn1nnnc1C)NC(=O)OC(C)(C)C]
and performing scheme 5; 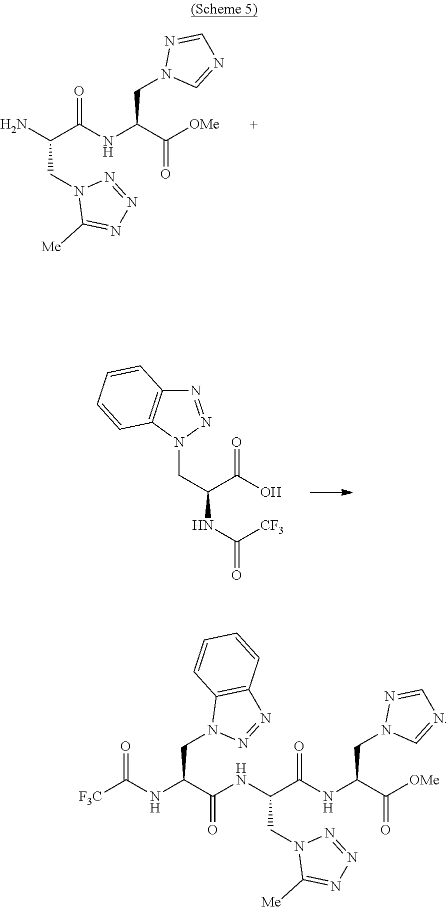 [SMILES: COC(=O)[C@H](Cn1cncn1)NC(=O)[C@@H](N)Cn1nnnc1C ; O=C(O)[C@H](Cn1nnc2ccccc21)NC(=O)C(F)(F)F ; COC(=O)[C@H](Cn1cncn1)NC(=O)[C@H](Cn1nnnc1C)NC(=O)[C@H](Cn1nnc2ccccc21)NC(=O)C(F)(F)F]


SEQUENCE LISTING (1 sequence)

SEQ ID NO: 1 - protein, 7 aa, synthetic construct
NLLMAAS